(19) United States
(12) Patent Application Publication
(10) Pub. No.: US 2026/0257186 A1
(43) Pub. Date: Sep. 3, 2026

(54) Tunable Structure of Biodegradable Silk-Based Microcapsules for Soluble and Insoluble Payload Delivery

(71) Applicants: MASSACHUSETTS INSTITUTE OF TECHNOLOGY, Cambridge, MA (US); BASF SE, Ludwigshafen am Rhein (DE)

(72) Inventors: Benedetto MARELLI, Lexington, MA (US); Muchun LIU, Cambridge, MA (US); Pierre-Eric MILLARD, Ludwigshafen (DE); Henning URCH, Limburgerhof (DE); Ophelie ZEYONS, Ludwigshafen (DE); Rupert KONRADI, Tarrytown, NY (US); Bernd OSCHMANN, Ludwigshafen (DE); Yagmur YEGIN, Cambridge, MA (US)

(21) Appl. No.: 18/713,968

(22) PCT Filed: Nov. 28, 2022
(86) PCT No.: PCT/US2022/080497

§ 371 Date: May 28, 2024

Related U.S. Application Data
(60) Provisional application No. 63/283,921, filed on Nov. 29, 2021.

(51) Int. Cl. B01J 13/04; A61K 8/11; A61K 8/64; A61Q 19/00; B01J 13/08; B01J 13/20; C05G 5/35

(52) U.S. Cl. CPC B01J 13/043; A61K 8/11; A61K 8/64; A61Q 19/00; B01J 13/08; B01J 13/20; C05G 5/35; A61K 2800/10; A61K 2800/412; A61K 2800/624

(57) ABSTRACT

The present disclosure provides microcapsules comprising a silk fibroin polypeptide and one or more active payload, materials, and formulations comprising the microcapsules of the disclosure. Also provided are methods of preparing and methods of using the microcapsules of the disclosure.

### CROSS REFERENCE TO RELATED APPLICATIONS

This application claims the benefit of U.S. Provisional Patent Application No. 63/283,921, filed Nov. 29, 2021, the contents of which are hereby incorporated in their entirety.

### STATEMENT REGARDING SEQUENCE LISTING

This application contains a Sequence Listing which is submitted electronically and is hereby incorporated by reference in its entirety. The sequence listing submitted herewith is contained in the XML filed created Nov. 28, 2022 entitled “21-1527-WO_SequenceListing.xml” and is 2 KB in size.

### BACKGROUND OF THE DISCLOSURE

From 1950 to 2015, the global production of plastics increased explosively from 2 to 380 million metric tons, resulting in an accumulation of plastic waste in the environment on an upward trajectory. Nowadays, plastic waste can be found in most of the physical environments on the planet (e.g., aquatic and terrestrial), as well as in the biological systems of living organisms that dwell in these areas. The high durability and resistance of plastics that once contributed to their key functional roles is now causing a huge degradation problem in their current role of waste. The striking facts and extensive reports of plastic pollution have fostered a phenomenal public perception of environmental risks, and thus prompted the public to demand quicker and decisive actions. Globally, a number of legislations have been successfully passed, including phase down/out on single-use plastic bags and bans on intentionally-added microplastics.

However, the plastics universe contains a wide spectrum of polymers with different composition, size, and functions rather than one homogeneous component, which results in different applications and therefore life cycles. The plastic leakages along the life-cycle paths are from sources including: 1) mismanaged plastic waste (e.g., uncollected packaging), 2) mechanical abrasion (e.g., tire wear), 3) plasticulture waste (e.g., uncollected mulch films), and 4) primary microplastics lost through environmental applications or consumption (e.g., herbicide microcapsules and cosmetic beads). Strategies to reduce the plastic pollution should be specific and with measurable goals. Potential solutions include enforceable waste management and education, waste disposal flow improvement, innovative strategies on recycling, and use of sustainable substitutions. For microplastics targeted in open use, such as environmental applications and household consumptions as stated above in scenario 3, it is difficult to avoid their release into the environment. Replacing these conventional, non-degradable microplastics (polyurea/melamine formaldehyde, polyurethane, polyamides, acrylic copolymers, etc.) with biodegradable counterparts is therefore precise and reasonable.

There exists a need for an effective biodegradable microcapsule that retains the release control function (slow discharge of core actives through shell materials) and uses a simple manufacturing process, with biodegradability as an add-on.

### SUMMARY OF THE DISCLOSURE

One aspect of the disclosure provides a microcapsule comprising a silk fibroin polypeptide and an active payload material. The payload material is present in an amount in the range of about 10% to about 50% by weight, based on solid weight of the microcapsule. In the microcapsule of the disclosure, the silk fibroin polypeptide forms a spherical shell having a smooth surface morphology. In addition, in the microcapsule of the disclosure the payload material is dispersed within the silk fibroin polypeptide shell.

The active payload material of the disclosure is not particularly limiting and may be water-soluble material or water-insoluble material (lipophilic liquid or insoluble solid).

Another aspect of the disclosure provides a monodisperse microcapsule composition. Such compositions comprise a population of microcapsules as described herein. The median diameter d50 may be in the range of 5 μm to 50 μm, where d10 is no less than 20% of d50 and d90 is no more than 500% of d50.

In certain embodiments, a monodisperse microcapsule composition of the disclosure is a plant cultivation composition. For example, in such a composition, the payload material is a pesticide (e.g. an herbicide, a fungicide, or an insecticide). In another example, the payload material is a fertilizer, a supplement, or another agrochemical. Another aspect of the disclosure provides a method of improving plant growth, the method comprising applying an effective amount of the plant cultivation composition to seed bed, soil, or plant (such as plant foliage).

In certain embodiments, a monodisperse microcapsule composition of the disclosure is a personal care composition. For example, in such composition, the payload material is a cosmetically active agent or a pharmaceutically active agent.

One aspect of the disclosure provides methods of preparing a microcapsule as described herein. In one embodiment, the method includes: (a) mixing a silk fibroin polypeptide and a payload material in an aqueous solution to obtain a mixture; (b) feeding and atomizing said mixture into a drying chamber to obtain droplets; and (c) drying said droplets to microcapsules. In another embodiment, the method includes: (a) mixing a silk fibroin polypeptide and a payload material in an aqueous solution to obtain a mixture; (b) feeding and atomizing said mixture into a cold fluid to obtain frozen droplets; and (c) drying said droplets to microcapsules. In a further embodiment, the method includes: (a) creating an organic phase composed of the one or more of active payload materials, optionally dissolved in a non-aqueous solvent that is immiscible with water; and (b) emulsifying the organic phase in water containing silk fibroin polypeptide to obtain microcapsules.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiment(s) of the disclosure and, together with the description, serve to explain the principles and operation of the disclosure.

FIG. 1 provides a conceptual overview; structural manipulation of regenerated silk fibroin suspension into microcapsules and their potential applications. FIG. 1A: Fabrication of regenerated silk fibroin (RSF) aqueous suspension from B. Mori cocoons. FIG. 1B: Secondary structure of silk fibroin (SF) can be transformed from random coil to beta sheet, resulting in increased crystallinity. FIG. 1C: Left, schematic of tunable structures of microcapsules, including smooth and crumpled surfaces, matrix and multi-domain structures. Red sphere. SF matrix: blue object, active. Right, proposed non-exhaustive and non-limiting applications of silk-based microcapsules with different structures.

FIG. 2 provides structure and chemical composition of silk fibroin and a water-soluble active Vitamin C (SF/VC) microcapsules fabricated from spray freeze drying (SFD) and spray drying (SD) methods. FIG. 2A: Left, Surface morphology of SF/VC microcapsules fabricated from SFD method, wt % ratio of SF:VC=10:5. Scale bar, 50 μm. Right, high-magnification view of a SF/VC microcapsule, showing a spherical shape with porous surface. Scale bar, 10 μm. FIG. 2B: Cross-section of a SFD SF/VC microparticle. Scale bar, 10 μm. FIG. 2C: Size distribution of SF/VC microcapsules from SFD and SD methods. FIG. 2D: Left, Surface morphology of SF/VC microcapsules (with 1% Tween 20) fabricated from SD method, wt % ratio of SF:VC:T=10:5:1. Scale bar, 10 μm. Right, high-magnification view of as-prepared microcapsule, showing a spherical shape with smooth surface. Scale bar, 2 μm. FIG. 2E: Cross-section of a SD SF/VCf microparticle, scale bar, 2 μm. FIG. 2F: FTIR results of SF/VC microparticles from SD methods, with and without ethanol treatment.

FIG. 3 illustrates an effect of composition on the colloidal behavior of silk-based suspensions and morphology of SF/VC microcapsules. FIG. 3A: Surface morphologies of SFD microparticles using different active and additives (10% SF, 10% SF-5% VC, 10% SF-10% VC, 10% SF-10% VC-0.56M NaOH and 10% SF-10% VC-1% Tween20). Scale bar, 10 μm. FIG. 3B: Surface morphology of SD microparticles with different composition (10% SF, 10% SF-5% VC, and 10% SF-5% VC-1% Tween20). Lower panel, surface morphology of SD microparticles after 90% ethanol soaking for 1 hr followed with overnight drying. Scale bar, 2 μm. FIG. 3C: Size and pH of different SF aqueous suspensions with different compositions (10% SF, 10% SF-5% VC, 10% SF-10% VC, 10% SF-10% VC-0.56M NaOH and 10% SF-10% VC-1% Tween20).

FIG. 4 provides structure and chemical composition of SF/Saflufenacil (SF/Saf) microcapsules fabricated from SD method. FIG. 4A: Top, Surface morphology of SD SF/Saf microcapsules (after ethanol treatment), wt % ratio of SF:Saf=5:5. Scale bar, 10 μm. Bottom, high-magnification view of a SD SF/Saf microcapsule (after ethanol treatment), showing a crumpled structure. Scale bar, 1 μm. FIG. 4B: Top, cross-section of a SD SF/Saf microcapsule (after ethanol treatment), showing a core-shell, multi-domain structure. Scale bar, 5 μm. Bottom, high-magnification view of fractured surface of a SD SF/Saf microcapsule, the irregular Saf crystals are embedded in a SF shell. Scale bar, 1 μm. FIG. 4C: Elemental analysis of SD SF/Saf microcapsules. The atomic percentage of F is much lower than that of Saf, indicating a multi-domain structure with Saf embedded in silk shell. FIG. 4D: Elemental analysis of a Saf base. FIG. 4E: Size distribution of SD SF/Saf microcapsules. FIG. 4F: FTIR results of SF/Saf microparticles with and without ethanol treatment.

FIG. 5 provides the release and biodegradation behaviors of silk-based microcapsules. FIG. 5A: Beta sheet percentage of SF microcapsules from SFD and SD methods, under different ethanol treatment time. FIG. 5B: Release behaviors of SF/VC microcapsules fabricated from two methods. The microcapsules are treated with ethanol. FIG. 5C: Release behaviors of SF/Saf microcapsules with different beta sheet percentages. FIG. 5D: Biodegradation profile of spray freeze dried and spray dried SF microcapsules.

FIG. 6 shows photographs of SF-based emulsions with different dibutyl adipate (DA), ethanol (Et) contents. FIG. 6A: fresh SF emulsions marked with different DA and Et wt % at day 0. Pre (the first SF) and post (the second) SF % is indicated in the table. FIG. 6B: aged emulsions marked with different DA and Et wt % at day 4. Pre (the first SF) and post (the second) SF % is indicated in the table.

FIG. 7 illustrates morphology and thermal stability of SF/DA emulsion (with final 18.1% solid (SF0.8-DA16.5-SF0.8)+8.3% EtOH). FIG. 7A: optical images of SF/DA emulsions of initial 0.8% SF and 8.3% ethanol, after adding complementary 0.8% SF, and after 4 days of aging. Scale bar, 5 μm. FIG. 7B: Thermal stability results of fresh and aged SF/DA emulsions. FIG. 7C: The table of remaining solid content of emulsions after the first (105° C. for 2 hrs) and second (130° C. for 1 hr) thermal treatments.

FIG. 8 illustrates morphology and thermal stability of SF/DA emulsion (with final 18.1% solid (SF0.8-DA 16.5-SF0.8)+0% EtOH). FIG. 8A: optical images of SF/DA emulsions of initial 0.8% SF and no ethanol, after adding complementary 0.8% SF, and after 4 days of aging. Scale bar, 5 μm. FIG. 8B: Thermal stability results of fresh and aged SF/DA emulsions. FIG. 8C: The table of remaining solid content of emulsions after the first (105° C. for 2 hrs) and second (130° C. for 1 hr) thermal treatments.

FIG. 9 illustrates morphology and thermal stability of SF/DA emulsion (with final 26.6% solid (SF0.8-DA25.0-SF0.8)+8.3% EtOH). FIG. 9A: optical images of SF/DA emulsions of initial 0.8% SF and 8.3% ethanol, after adding complementary 0.8% SF, and after 4 days of aging. Scale bar, 5 μm. FIG. 9B: Thermal stability results of fresh and aged SF/DA emulsions. FIG. 9C: The table of remaining solid content of emulsions after the first (105° C. for 2 hrs) and second (130° C. for 1 hr) thermal treatments.

FIG. 10 illustrates morphology and thermal stability of SF/DA emulsion (with final 26.6% solid (SF0.8-DA25.0-SF0.8)+0% EtOH). FIG. 10A: optical images of SF/DA emulsions of initial 0.8% SF and no ethanol, after adding complementary 0.8% SF, and after 4 days of aging. Scale bar, 5 μm. FIG. 10B: Thermal stability results of fresh and aged SF/DA emulsions. FIG. 10C: The table of remaining solid content of emulsions after the first (105° C. for 2 hrs) and second (130° C. for 1 hr) thermal treatments.

FIG. 11 illustrates morphology and thermal stability of SF/DA emulsion (with final 16.7% solid (SF0.4-DA15.2-SF1.1)+0% EtOH). FIG. 11A: optical images of SF/DA emulsions of initial 0.4% SF and no ethanol, after adding complementary 1.1% SF, and after 4 days of aging. Scale bar, 5 μm. FIG. 11B: Thermal stability results of fresh and aged SF/DA emulsions. FIG. 11C: The table of remaining solid content of emulsions after the first (105° C. for 2 hrs) and second (130° C. for 1 hr) thermal treatments.

FIG. 12 illustrates thermal stability of DA emulsion (DA mixed with pure water without adding SF). Left, thermal stability results of DA emulsion. Right, the table of remaining solid content of emulsions after the first (105° C. for 2 hrs) and second (130° C. for 1 hr) thermal treatments.

FIG. 13 illustrates the thermal stability of SF/DA emulsion with final 26.75% solid (SF0.75-DA25-SF1) and sonicated either for 5, 8 of 15 sec after the first (105° C. for 2 hrs) and second (130° C. for 1 hr) thermal treatments.

FIG. 14 illustrates the thermal stability of SF/DA+Tinosorb® S emulsion with final 26.75% solid (SF0.75-Tinosorb® S10-DA15-SF1) after the first (105° C. for 2 hrs) and second (130° C. for 1 hr) thermal treatments.

FIG. 15 provides schematic of SD and SFD processes. The RSF aqueous suspension was well mixed with different core actives. For SFD method, the SF/VC mixed microdroplets were generated using a vortex ultrasonic spray nozzle and then collected in a liquid nitrogen bath followed with lyophilization. For SD method, the SF/VC mixed suspension was air sprayed using a two-fluid nozzle under compressed air, then dried out in rapid heated air.

FIG. 16 illustrates morphologies of SD SF/VC microcapsules. The feed suspension contains 10% SF and 5% VC. The as-prepared microcapsules show collapsed and crumpled structures. Scale bars, 10 μm (left), 1 μm (middle and right).

FIG. 17 illustrates morphology of SFD microcapsules before and after ethanol treatment. After 90% ethanol treated for 1 hr, the SFD SF/VC microcapsules deformed and fused to each other. Scale bar, 20 μm.

FIG. 18 illustrates morphology of Saf base after probe sonication. A Saf base was vigorously sonicated to around 1 μm in size. Scale bar, 1 μm.

FIG. 19 shows calibration curves of concentration-absorption by UV-Vis. FIG. 19A: VC. FIG. 19B: Saf.

FIG. 20 illustrates release profiles of SD SF/VC, SF/VC/T microcapsules with SF beta sheet of 25%, and SFD SF/VC microcapsules with SF beta sheet of 20%.

### DETAILED DESCRIPTION

Open use of synthetic plastic-based microcapsules such as agricultural applications and household consumptions make it inevitable to release microplastic waste to the environment. Conventional microplastics are known to last for a long time, they travel in a variety of ecosystems, and accumulate in living organisms along food chains. Continuous use of such microplastics has raised tremendous concerns among the public. It is desired and reasonable to replace these non-degradable microplastics targeted in open use situations with biodegradable counterparts.

Among the studied biodegradable polymers, such as polylactides, chitosan, cellulose, alginates, lignin, etc., the inventors found that silk protein is an outstanding candidate due to its food-grade safety, case of processing, and tunability in chemical and physical properties. Thus, the present disclosure provides a biopolymer-based encapsulation technology that uses silk protein to create a series of biodegradable microcapsules for soluble and insoluble payloads. Adjusting feed suspensions and fabrication processes tunes the morphology, structure, release and biodegradation kinetics of said microcapsules.

The present inventors have transformed the secondary structure of silk protein from hydrophilic random coil to hydrophobic beta sheet by exposure to organic solvents, heat, or mechanical stress. The structural transition function is specifically suitable for silk protein to encapsulate different core actives, including hydrophilic (e.g., Vitamin C in cosmetic beads), lipophilic (e.g., hydrophobic pesticide) and insoluble (e.g., solid herbicides in agricultural capsules) payloads. Besides, the tunable crystallinity by controlling beta sheet percentage can adjust the mechanical properties and degradation time, making silk a strong candidate for diverse applications (L. Gasperini et al., Journal of The Royal Society Interface 2014, 11, 20140817; R Elia et al., Journal of Coatings Technology and Research 2015, 12, 793; S. S. Deveci, G. Basal, Colloid and Polymer Science 2009, 287, 1455).

In certain embodiments, the microcapsule of the disclosure as described herein is a porous sphere. “Porosity” as used herein, refers to a measure of void spaces in a material and is a fraction of volume of voids over the total volume, as a percentage between 0 and 100%. A determination of a porosity is known to a skilled artisan using standardized techniques, for example mercury porosimetry and gas adsorption (e.g., nitrogen adsorption). In certain embodiments, the microcapsules of the disclosure have a porosity in the range of about 1% and 50%, such as in the range of about 1% and 25%, or 1% and 10%, or 10% and 50%. In certain embodiments, the microcapsules of the disclosure have a porosity of at least about 50%, such as at least about 60%, at least about 70%, etc. In certain embodiments, the microcapsules of the disclosure are not porous.

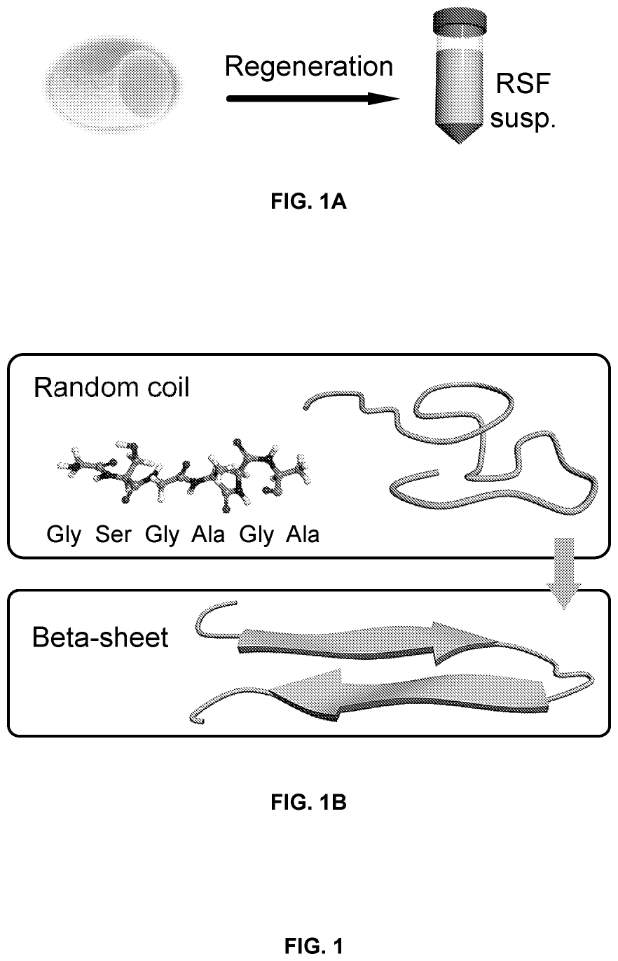

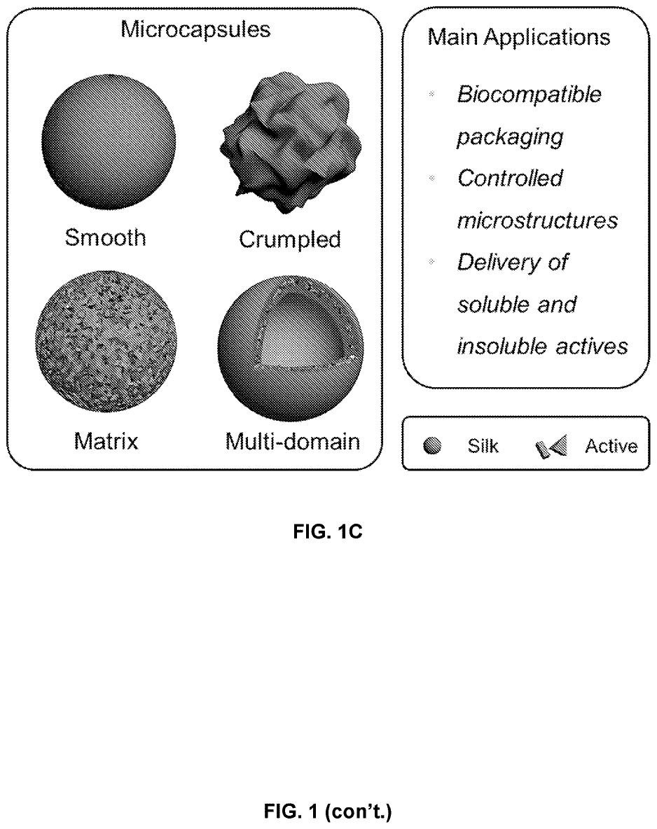

The present disclosure demonstrates the use of microencapsulation using silk fibroin (SF) as shell materials for open use applications (FIG. 1), includes disclosure pertaining to morphology control using different fabrication methods and composition of feed suspensions, and demonstrates release kinetics and biodegradation profiles. In a non-limiting example, silk-based microcapsules with either water-soluble Vitamin C (VC, ascorbic acid) or water-insoluble Saf, an herbicide) as core actives were prepared. Spray drying or spray freeze drying methods were selected due to simple process, accurate control on loading, and high yield. SF/VC (weight ratio: 10:5) and SF/Saf (weight ratio: 5:5) were obtained in gram-scale.

Silk fibroin of the disclosure includes a wide variety of silk fibroin polypeptide, fragments thereof, including preparations extracted from native sources, produced recombinantly, or chemically synthesized. For example, silk fibroin useful for the present invention may be that produced by a number of species, including, without limitation: Antheraea mylitta, Antheraea pernyi, Antheraea yamamai, Galleria mellonella. Bombyx mori, Bombyx mandarina, Galleria mellonella, Nephila clavipes, Nephila senegalensis, Gasteracantha mammosa, Argiope aurantia, Araneus diadematus, Latrodectus geometricus, Araneus bicentenarius, Tetragnatha versicolor, Araneus ventricosus, Dolomedes tenebrosus, Euagrus chisoseus, Plectreurys tristis, Argiope trifasciata, and Nephila madagascariensis.

In some embodiments, silk fibroin of the disclosure is obtained from a solution containing a dissolved silkworm silk or spider silk. For example, in some embodiments, silkworm silk fibroins are obtained, from the cocoon of Bombyx mori. In some embodiments, spider silk fibroins are obtained, for example, from Nephila clavipes. In some embodiments, silk fibroins of the disclosure are obtained from a solution containing a genetically engineered silk harvested from bacteria, yeast, mammalian cells, transgenic animals or transgenic plants.

Different types of silk produced by different organisms may have different amino acid compositions, but their silk fibroin share certain structural features. Generally, silk fibroin structure is a sequence of amino acids that is characterized by usually alternating glycine and alanine, or alanine alone. Such configuration allows fibroin molecules to self-assemble into a beta sheet conformation. These “Ala-rich” hydrophobic blocks are typically separated by segments of amino acids with bulky side-groups (e.g., hydrophilic spacers). For example, silk % worm fibroin protein consists of layers of antiparallel beta sheets whose primary, structure mainly consists of the recurrent amino acid sequence GAGAGS (SEQ ID NO. 1).

The silk fibroin may be crosslinked after encapsulation, or it may be not crosslinked. In certain embodiments, the silk fibroin is obtained by fermentation.

In certain embodiments, the silk fibroin of the disclosure as described herein may be a polypeptide, with or without one or more sequence variations, as compared to the native or wild type silk fibroin. For example, in some embodiments, the silk fibroin of the disclosure may show at least 51% overall sequence identity as compared to the native or wild type silk fibroin, e.g., such as the silkworm silk fibroins obtained from the cocoon of Bombyx mort. For example, in certain embodiments, the silk fibroin of the disclosure may show at least 55%, 60%, 65%, 70%, 75%, 80%, 85%, 90%, 91%, 92%, 93%, 94%, 95%, 96%, 97%, 98%, or 99% overall sequence identity as compared to the native or wild type silk fibroin.

In some embodiments, the silk fibroin of the disclosure as described herein has the beta sheet content of not greater than about 35%, e.g., not greater than about 30%, not greater than about 25%, not greater than about 20%, not greater than about 15%, or not greater than about 10%.

In some embodiments, the silk fibroin of the disclosure as described herein has the beta sheet content in the range of 10% to 80%. For example, in certain embodiments, the beta sheet content is in the range of 10% to 60%, or 10% to 50%, or 10% to 40%, or 10% to 30%, or 10% to 20%. In certain embodiments, the beta sheet content is in the range of 20% to 80%, or 20% to 50%, or 20% to 40%, or 20% to 30%. In certain embodiments, the beta sheet content is in the range of 25% to 80%, or 25% to 50%, 25% to 40%, or 25% to 35%, or 25% to 30%.

In some embodiments, the silk fibroin of the disclosure as described herein has the beta sheet content of at least about 10%. For example, the beta sheet content is at least about 15%, or at least about 20%. In some embodiments, the silk fibroin of the disclosure as described herein has the beta sheet content of at least about 25%, for example at least about 30%, at least about 35%, or at least about 40%.

In certain embodiments, the beta sheet content of the microcapsules of the invention can be increased with any suitable treatment, e.g. methanol, ethanol, propanol, glycol, acetone, salt solution or any other treatment (such as a physical process using ultra-sound).

The silk fibroin of the disclosure as described may have an average molecular weight in the range of about 15 kDa and about 400 kDa. In some embodiments, suitable silk fibroins include, but are not limited to, silk fibroin polypeptides having an average molecular weight in the range of about 15 kDa and about 300 kDa, about 15 kDa and about 200 kDa, about 15 kDa and about 150 kDa, about 15 kDa and about 100 kDa, about 25 kDa and about 400 kDa, about 25 kDa and about 300 kDa, about 25 kDa and about 200 kDa, about 25 kDa and about 150 kDa, about 25 kDa and about 100 kDa, about 50 kDa and about 400 kDa, about 50 kDa and about 300 kDa, about 50 kDa and about 200 kDa, about 50 kDa and about 150 kDa, about 100 kDa and about 400 kDa, about 100 kDa and about 300 kDa, about 100 kDa and about 250 kDa, about 100 kDa and about 200 kDa, about 200 kDa and about 400 kDa, about 200 kDa and about 300 kDa, or about 300 kDa and about 400 kDa.

The disclosure also provides a monodisperse microcapsule composition comprising a population of microcapsules of the disclosure as described herein having uniform average diameter. Thus, in certain embodiments, the distribution of the average diameters follows a Gaussian profile.

For example, in certain embodiments of the microcapsule composition of the disclosure, the population of the microcapsules may have a relatively narrow diameter distribution. As the person of ordinary skill in the art will appreciate, the diameter distribution can be characterized by d50, d10 and d90 values, where d50 is the median diameter, d10 is the diameter at the 10^{th} percentile of microcapsules ranked by size, and d90 is the diameter at the 90^{th} percentile of microcapsules ranked by size. In certain embodiments, the microcapsules of a particular composition as otherwise described herein have a d50 value in the range of 1 μm to 50 μm; e.g., 1 μm to 50 μm, or 3 μm to 50 μm, or 5 μm to 50 μm, or 5 μm to 40 μm, or 10 μm to 40 μm, or 20 μm to 40 μm. In certain embodiments, the microcapsules of a particular composition as otherwise described herein have a d50 value in the range of 1 μm to 50 μm; e.g. 1 μm to 40 μm, or 2 μm to 30 μm, or 3 μm to 20 μm, or 3 μm to 25 μm, or 3 μm to 15 μm. In certain embodiments, the microcapsules of a particular composition as otherwise described herein have a d50 value in the range of 15 μm to 50 μm: e.g., 15 μm to 40 μm. In certain embodiments, the microcapsules of a particular composition as otherwise described herein have a d50 value in the range of 0.1 μm to 50 μm, e.g. 1 μm to 15 μm.

The payload material, or active component of the microcapsules of the invention may be any suitable material for the desired application, including, in non-limiting examples, active agents for use in cosmetic or pharmaceutical purposes, or as a pesticide, fertilizer, supplement, or other agrochemical. In certain non-limiting examples, the payload materials may be pesticides, synergists, plant health agents, repellants, biocides, phase-change materials, pharmaceuticals, cosmetic ingredients (like fragrances, perfumes, vitamins, essential oils, plant extracts), nutrients, food additives (like vegetable oils, marine oils, vitamins, aromas, antioxidants, essential oils, plant extracts), pheromones, catalysts, or combinations thereof.

The payload material may be a water-soluble or a water-insoluble material (i.e. a lipophilic liquid or an insoluble solid).

The active payload material may be hydrophilic or it may be lipophilic. The active payload material may be solid at room temperature and water insoluble, and such a payload material may be dissolved in a non-aqueous solvent that is immiscible with water.

In certain embodiments of the invention, the active payload is present in the microcapsules in the range of about 10% to about 98% by weight, based on solid weight of the microcapsule, or may be present within any range therein, for example about 10% to about 70%, about 20% to about 50%, about 10% to about 50%, about 30% to about 50%, about 35% to about 45%, about 25% to about 50%, about 10% to 98%, about 20% to 95%, about 25% to 40%, about 25% to 35%, above 50%, or above 70%.

The ratio of silk fibroin polypeptide to active payload material may be from about 10:1 to about 1:50, such as about 3:1 to about 1:50, about 3:1 to about 1:25, about 3:1 to about 1:10, about 3:1 to about 1:5, or about 3:1 to about 1:3.

The microcapsules of the invention may be a core-shell microcapsule having a shell of silk fibroin polypeptide and a core composed of the one or more payload materials. In certain embodiments, the one or more active payload materials may be dissolved in a non-aqueous solvent that is immiscible with water.

As used herein “non-aqueous solvent” is a solvent that has miscibility in water of less than 5 g/L.

In certain embodiments, the microcapsules of the invention may be a microsphere with at least one active substance dispersed within the silk fibroin polypeptide payload. In certain embodiments, the microcapsule may be a hollow sphere.

The microcapsules of the invention may be prepared by any suitable method. In one embodiment, the microcapsules of the invention are prepared by (a) mixing a silk fibroin polypeptide and one or more of active payload materials in an aqueous solution to obtain a mixture; (b) feeding and atomizing said mixture into a drying chamber to obtain droplets; and (c) drying said droplets to microcapsules. In another embodiment, they are prepared by (a) mixing a silk fibroin polypeptide and one or more of active payload materials in an aqueous solution to obtain a mixture; (b) feeding and atomizing said mixture into a cold fluid to obtain frozen droplets; and (c) drying said droplets to microcapsules.

In a further embodiment, the microcapsules of the invention may be prepared by (a) create creating an organic phase composed of the one or more of active payload materials, optionally dissolved in a non-aqueous solvent that is immiscible with water; and (b) emulsifying the organic phase in water containing silk fibroin polypeptide to obtain microcapsules.

In certain embodiments, the microcapsules of the invention are formulated as a microemulsion.

The present disclosure also provides formulations comprising the microcapsules of the invention. In such formulations, the microcapsules may be present as dispersed particles in an aqueous medium. In certain non-limiting examples, such formulations comprise 1 to 50 wt %, preferably 5 to 45 wt %, more preferably 10 to 40 wt % of said one or more active substances.

The present disclosure also provides methods of using the microcapsules as described herein, or the formulations comprising the microcapsules, in various applications, including, but not limited to, agrochemical applications (e.g. crop protection, agricultural non-crop applications, seed treatment), pharmaceutical applications, public health, personal care applications (such as cosmetic applications), construction applications, textile applications, human or animal nutrition applications, chemical process applications, adhesives and sealants, paints and coatings, building and construction materials, self-healing materials, tobacco industry, and household applications. The present disclosure also provides for use of the microcapsules as described herein, or the formulations comprising the microcapsules, in various applications, including those described immediately above.

The agrochemical applications include, but are not limited to, controlling phytopathogenic fungi and/or undesired plant growth and/or undesired attack by insects or mites and/or for regulating the growth of plants. In the methods of the invention for such applications, the microcapsules of the invention, of formulations comprising the microcapsules, may be applied to the plants and/or to the soil, and may comprise allowing the microcapsules or formulation to act on the pests, their habitat, the plants to be protected from the pests, the soil, and/or on undesired plants and/or the useful plants and/or their habitat. The agrochemical applications also include methods improving plant growth comprising applying an effective amount microcapsules according to the invention, or formulations comprising same, to a seed bed, soil, and/or plant, such as plant foliage.

In certain embodiments, the microcapsules of the invention are formulated into seed coatings.

### EXAMPLES

The compositions and methods of the disclosure are illustrated further by the following examples, which are not to be construed as limiting the disclosure in scope or spirit to the specific methods and compounds described in them.

### Example 1: Materials and Methods

### Materials

B. Mori cocoons were purchased from a local silk farm in South Korea (Gokseong-gun. Jeollanam-do). Lithium bromide, sodium carbonate, ascorbic acid, sodium hydroxide solution 5.0 M, TWEEN® 20 10% (w/v) aqueous solution, sodium dodecyl sulfate, antifoam B emulsion (a 10% aqueous emulsion of polydimethylsiloxane), protease from Streptomyces griseus (Type XIV, ≥3.5 units mg⁻¹ solid, powder), and dibutyl adipate were purchased from Sigma-Aldrich. Gibcor™ PBS (10×), pH 7.4 was purchased from Thermo Fisher Scientific. Acetate buffer solution, pH 4.0 was purchased from VWR Chemicals BDH®. Saflufenacil, Pluronic PE 10500, and Tinosorb® S were kindly provided by BASF SE. All water was deionized (18.2 MΩ, milli-Q pore). All reagents were used as received without further purification.

### Methods

### Regeneration of Silk Fibroin Suspension

The regenerated silk fibroin was extracted from cocoons of B. mori following an established protocol (D. N. Rockwood et al., Nature Protocols 2011, 6, 1612.). In brief, silk cocoons were cut into pieces (2×2 cm²) and boiled in 0.02 M sodium carbonate solution for 120 min to remove the sericin coating. The degummed silk fibers were rinsed with Milli-Q water (18.2 MΩ cm) and dried overnight. The dried silk fibers were dissolved in 9.3 M lithium bromide for 4 hrs at 60° C. followed by dialysis against Milli-Q water in dialysis membranes (molecular weight cut-off: 3500 Da) for 3 days with frequent change of water. The dissolved silk fibroin suspension was then centrifuged to remove impurities (at 4800×g, 25 min×2). The final silk fibroin suspension was stored at 4° C. its concentration was determined gravimetrically.

### Fabrication of Silk-Based Microcapsules by Spray Drying

SF/VC microcapsule: 250 mL of 10% SF and 5% VC (or 10% SF, 5% VC and 1% Tween 20) aqueous suspension was well mixed and sprayed by a mini spray dryer SD-18A (LABFREEZ Instruments). The inlet temperature was set as 150° C., inlet air rate as 75% (maximum 330 m³ H⁻¹), feed rate of the peristaltic pump as 10% (maximum 2000 mL H⁻¹), and gas flow meter as 600 L H⁻¹. The outlet temperature was monitored as 80-90° C. The atomization was achieved by an air spray nozzle. The as-prepared microcapsules were collected and treated with 90% ethanol for 60 min.

SF/Saf microcapsule: A Saf base aqueous suspension was prepared with 20% of Saf solid powders, 0.6% sodium dodecyl sulfate, 0.2% antifoam B emulsion. The mixed suspension was thoroughly sonicated by a Branson Digital Sonifier SFX 550, with 20% of amplitude and 10 sec on/10 sec off pulse sonication for 2 hrs.

250 mL of 10% SF and 10% freshly made Saf base was well mixed and sprayed by a mini spray dryer SD-18A (LABFREEZ Instruments). The inlet temperature was set as 150° C., inlet air rate as 75% (maximum 330 m³ H⁻¹), feed rate of the peristaltic pump as 10% (maximum 2000 mL H⁻¹), and gas flow meter as 600 L H⁻¹. The outlet temperature was monitored as 80-90° C. The atomization was achieved by an air spray nozzle. The as-prepared microcapsules were collected and treated with 90% ethanol for 60 min.

### Fabrication of Silk-Based Microcapsules by Spray Freeze Drying

20 mL of 10% SF, 10% SF-5% VC, 10% SF-10% VC, 10% SF-10% VC-0.56M NaOH and 10% SF-10% VC-1% Tween20 aqueous suspensions were well mixed and slowly filtered using paper filters (5 μm pore size) to eliminate the bubbles. A Sono-tek ECHO ultrasonication generator with a 120 kHz vortex nozzle was used to spray SF-active-additive aqueous microdroplets at the rate of 3 mL min⁻¹. The rotational nitrogen gas for the vortex nozzle was set to 2 psi. The microdroplets were collected in a liquid nitrogen pool and followed with lyophilization.

### Fabrication of SF/DA Emulsion

Preparation of SF aqueous suspension: SF boiled for 4 hrs was prepared. The molecular weight decreases with the increased boiling time.

Preparation of SF/DA emulsion: 10 mL of 1% SF aqueous suspension was mixed well with a specific percentage of DA, followed with Is on/Is off pulse sonication for 5 sec, amplitude 15%. In case of treatment with ethanol, the obtained emulsion was dropwise added with ethanol under 1600 rpm stirring. The ethanol added emulsion was then dropwise added with additional 1% of SF aqueous suspension. A blank DA emulsion was prepared similarly with the absence of SF.

### Alternative Process

### Fabrication of SF/DA Emulsion 2

Preparation of SF aqueous suspension: SF boiled for 2 hrs was prepared.

Preparation of SF/DA emulsion: 4 mL of 0.75% SF aqueous suspension was mixed well with 25% DA, followed with is on/is off pulse sonication for either 5 sec or 8 sec or 15 sec, amplitude 15%. The obtained emulsion was dropwise added with additional 1% of SF aqueous suspension under 1600 rpm stirring.

### Fabrication of SF/DA+Tinosorb® S Emulsion

Preparation of DA+Tinosorb® S solution: The solution was prepared by mixing 60 wt % DA and 40 wt % Tinosorb® S. DA was warmed up to 65° C. Then, Tinosorb® S was added into warm DA and stirred until all Tinosorb® S completely dissolved. The organic solution was cooled to mom temperature.

Preparation of SF/DA+Tinosorb® S emulsion: 4 mL of 0.75% SF aqueous suspension was mixed well with 25% DA/Tinosorb® g S solution, followed with 1s on/1s off pulse sonication for 5 sec, amplitude 15%. The obtained emulsion was dropwise added with additional 1% of SF aqueous suspension under 1600 rpm stirring.

### Biodegradation of SF Microcapsules

The enzymatic degradation of SF microcapsules was done in PBS (1×, pH-7.4) solution at 37° C.

For control groups: around 7.5 mg of spray dried and spray freeze dried SF microcapsules (90% ethanol treated for 60 min) were immersed in 1 mL of PBS solution and put in a 37° C. oven. The sample was monitored for 10 days. Samples were washed with Milli-Q water (centrifuged at 20817×g, 10 min×4) and dried under 60° C. overnight before each weighing.

For experimental group: around 7.5 mg of spray dried and spray freeze dried SF microcapsules (90% ethanol treated for 60 min) were immersed in 1 mL PBS solution with 3.5 U mL⁻¹ protease IV from Streptomyces griseus and put in a 37° C. oven. The old solution was centrifuged out and new protease IV PBS solution was replenished daily. Samples were washed with Milli-Q water (centrifuged at 20817×g, 10 min×4) and dried under 60° C. overnight before each weighing. The mass of empty test vial, test vial with dried control sample and test vial with dried experimental sample were marked as m₀, m_{c} and mₚ. The weight remaining % was calculated as (mₚ−m₀ₚ)/(m_{c}−m_{0c})×100%.

### Release Kinetics

Release of SF/VC microcapsules: around 3 mg of different SF/VC microcapsules were dispersed in 20 mL of diluted acetate buffer solution (pH~4), wrapped in Al foil and under mild shaking (20 rpm). After certain time intervals, 0.75 mL of solution was taken for UV-Vis measurement to determine the concentration of released VC. The characteristic UV absorption peak position of VC is around 254 nm.

Release of SF/Saf microcapsules: around 5 mg of different SF/Saf microcapsules were dispersed in 50 mL of 10% Pluronic PE 10500 aqueous suspension, wrapped in Al foil and under 20 rpm mind shaking. After certain time intervals, 0.75 mL of suspension was taken for UV-Vis measurement to determine the concentration of released Saf. The characteristic UV absorption peak position of Saf is around 271 nm.

### Thermal Release of SF/DA Microemulsions

Around 2 mL of SF/DA emulsion (mass as mₑₘᵤₗₛᵢₒₙ) was added into Al pan (mass as m₀). The sample pan was then put in a 105° C. oven with fan for 2 hrs. After cooled down, the mass of treated sample pan was marked as m₁. The sample pan was put back to a 130° C. oven with fan for another 1 hr. After cooled down, the mass of sample pan was marked as m₂. The solid content % was calculated as (m₁−m₀)/mₑₘᵤₗₛᵢₒₙ×100, and the remaining content % after two-stage thermal treatment was calculated as (m₂−m₀)/mₑₘᵤₗₛᵢₒₙ×100. Experiments were run in duplicates.

### Statistical Analysis

All release and degradation tests were run in triplicates, and the standard errors for the analytical results were used to generate and present error bars.

### Characterization

The surface morphologies of microcapsules structures were investigated using a Zeiss Merlin High-resolution SEM operating at 0.8 kV for low-, medium- and high-resolution imaging. The elemental analysis was carried out on Zeiss Merlin SEM with energy-dispersive X-ray spectroscopy (EDS). Optical images of SF/DA emulsions were taken with a Nikon Eclipse TE2000-E optical microscope. The concentrations of released VC and Saf were determined by ultraviolet-visible (UV-Vis) spectroscopy on a VWR UV-1600PC spectrophotometer. Chemical structures of SF were studied with Fourier transform infrared (FTIR) spectroscopy from a Perkin Elmer Spectrum 65 spectrometer with the attenuated total reflection (ATR) accessory. The size of SF suspensions was studied using dynamic light scattering (DLS) on a Malvern Panalytical Zetasizer Nano-ZS. All suspensions were prepared in Milli-Q water. The data was analyzed on volume-based distribution.

### Example 2: Results and Discussion

Regenerated silk fibroin (RSF) was extracted from cocoons of B. Mori and dissolved into an aqueous suspension (FIG. 1A, experimental details in Methods). SF consists of a heavy (~390 kDa) and a light protein chain (~26 kDa) which are connected by disulfide linkage (K. Yamaguchi et al., Journal of Molecular Biology 1989, 210, 127; C.-Z. Zhou et al., Proteins: Structure, Function, and Bioinformatics 2001, 44, 119). The heavy protein chain is primarily composed of hydrophobic and robust GAGAGS (SEQ ID NO: 1) repeated sequences linked with hydrophilic amorphous amino acid linkers (R. L. Horan et al., Biomaterials 2005, 26, 3385). Raw SF from silk cocoons has high content of crystalline beta sheets thus is highly hydrophobic (X. Hu, D. Kaplan, P. Cebe, Macromolecules 2006, 39, 6161). We disconnected the linkage between amino acid domains and broke them into shorter blocks during boiling process. Then we broke the hydrogen bonding to using strong H-receptors and finally obtained a random coil aqueous suspension. Once exposed to physical or chemical triggers, the random coil of SF can then be transformed back to beta sheet (FIG. 1B). In this study we harnessed the folding behaviors of SF protein chains and prepared microcapsules with different surface morphologies and structures for open use applications (FIG. 1C). The fabrication process of spray dried (SD) and spray freeze dried (SFD) microcapsules is illustrated in FIG. 15 and in the methods section.

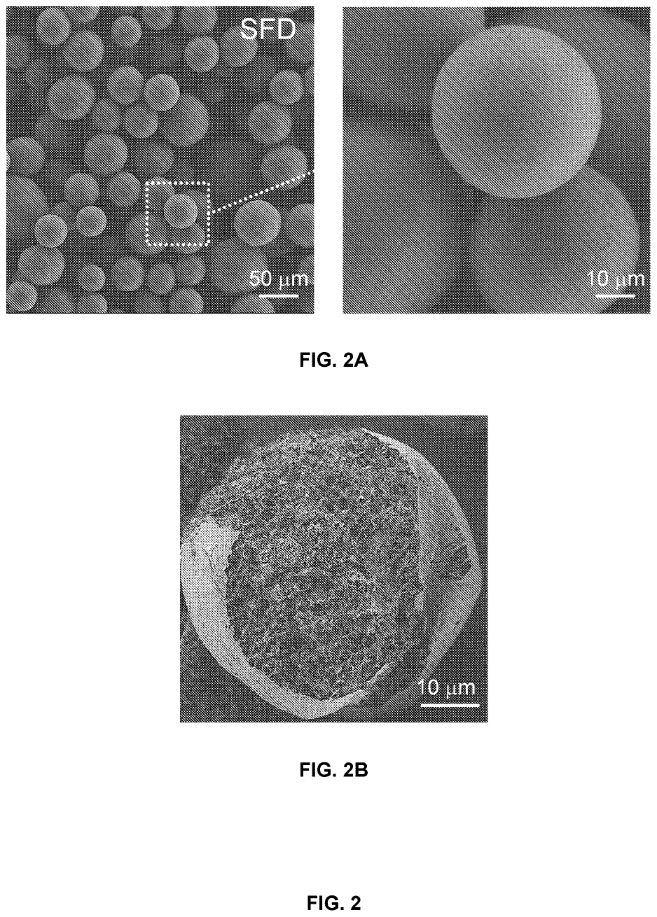

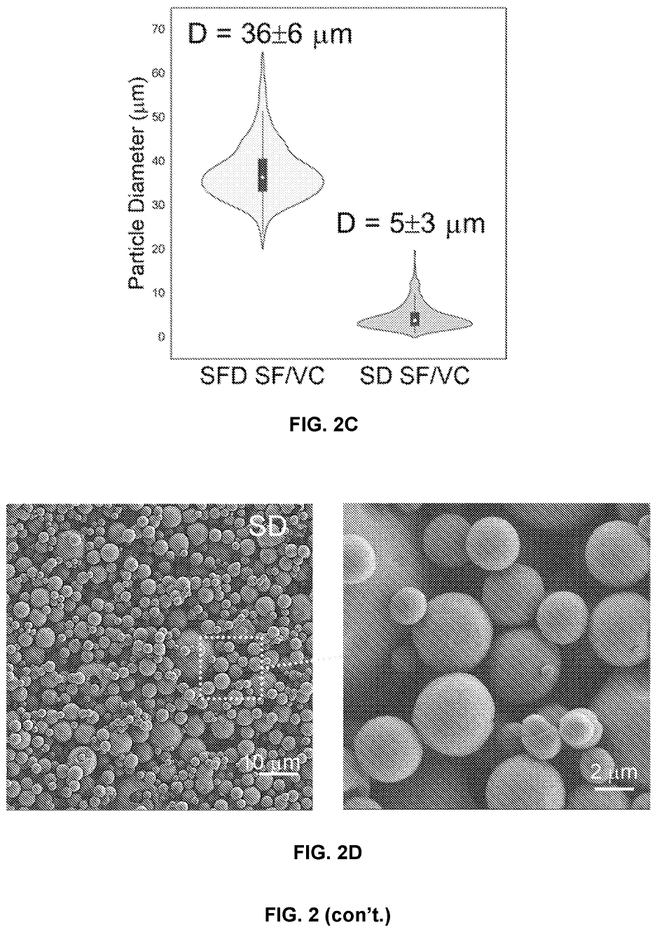

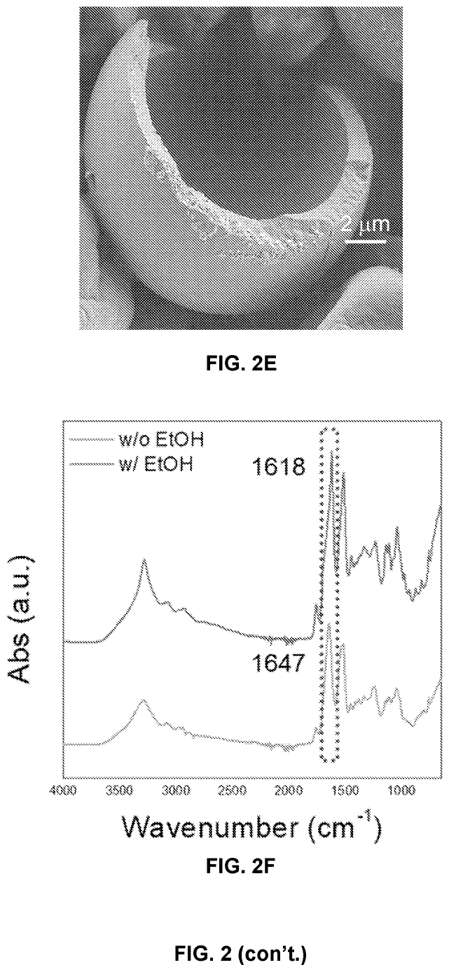

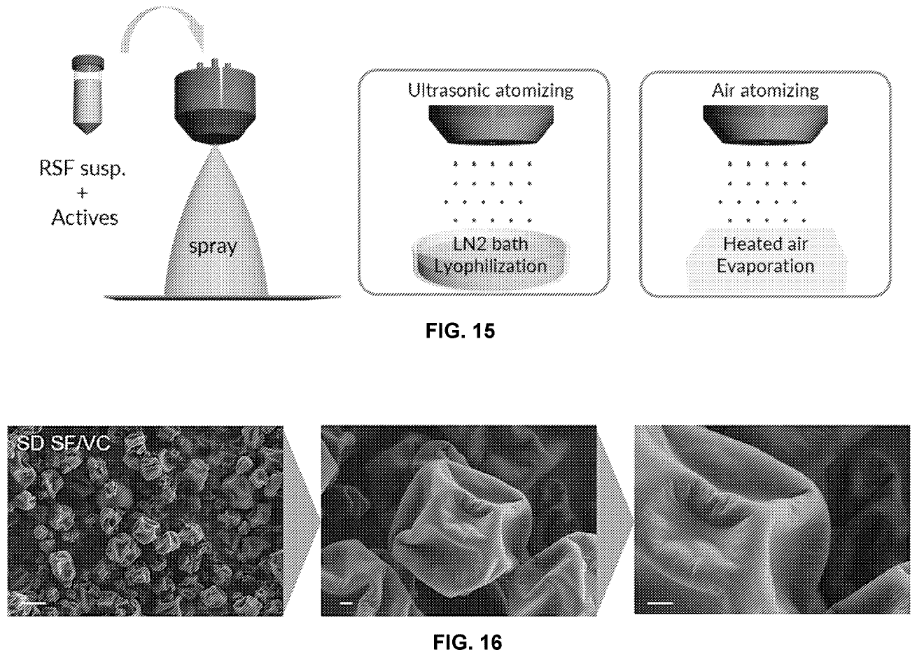

FIG. 2 presents a detailed characterization on the composition and structure of silk fibroin and a water-soluble active Vitamin C (SF/VC) microcapsule. For SFD SF/VC microcapsules, we first generated microdroplets of mixed SF/VC suspension using a vortex ultrasonic spray nozzle. The mechanical energy converted from ultrasound waves stimulates standing waves at the surface of nozzle, and then atomizes the feed suspension into fine microdroplets. The median diameter of the microdroplets is determined by the resonate frequency and therefore falls within a tight distribution (Sono-Tek). The feed microdroplets were collected in a liquid nitrogen bath followed with lyophilization. The SFD SF/VC (feed suspension contains 10% SF and 5% VC) microcapsules showed a spherical shape with a relatively smooth surface (FIG. 2A). When cut in half, the microcapsule showed a porous, sponge-like structure inside (FIG. 2B). FIG. 2B presents a size distribution of 36±6 μm. The SD silk fibroinNitamin C/Tween20 (SF/VCMT) and SD SF/VC microcapsules were prepared using a spray dryer, where the feed suspension was atomized through a two-fluid nozzle by compressed air then dried out in rapid heated air. The SD SF/VC (feed suspension contained 10% SF and 5% VC) microcapsules showed crumpled surfaces with collapsed structures (FIG. 16), while SF/VC/T (feed suspension contains 10% SF, 5% VC and 1% Tween20) microcapsules were also microspheres with dense surfaces (FIG. 2D), with a size distribution of 5±3 μm. To enhance uniformity and reduce uncertainty from complex morphology, we added 1% of a surfactant Tween20 to restore the spherical structure. The SD SF/VC/T microcapsules are hollow microspheres with dense surfaces (FIG. 2E). No phase separation of SF and VC was observed by SEM; we anticipated that water-soluble VC and SF would form a mixed matrix structure. An ethanol treatment was introduced to as-prepared SD and SFD microcapsules. As shown in FIG. 2F, the amide I region centered at 1647 shifted to 1618 cm⁻¹ after exposure to ethanol, which indicates a conformational transition of SF chains from random coil to beta sheet conformation. The effect of composition/treatment and possible causes of the morphological difference are discussed below.

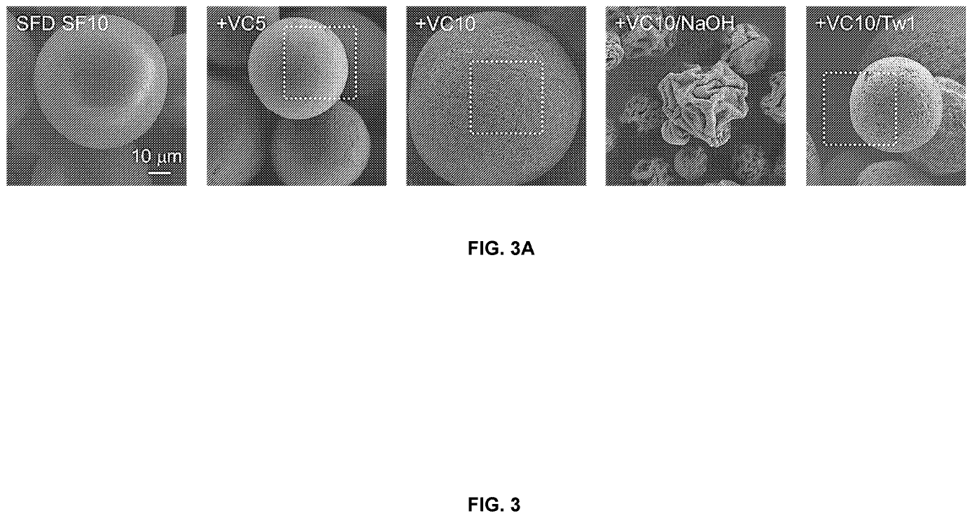

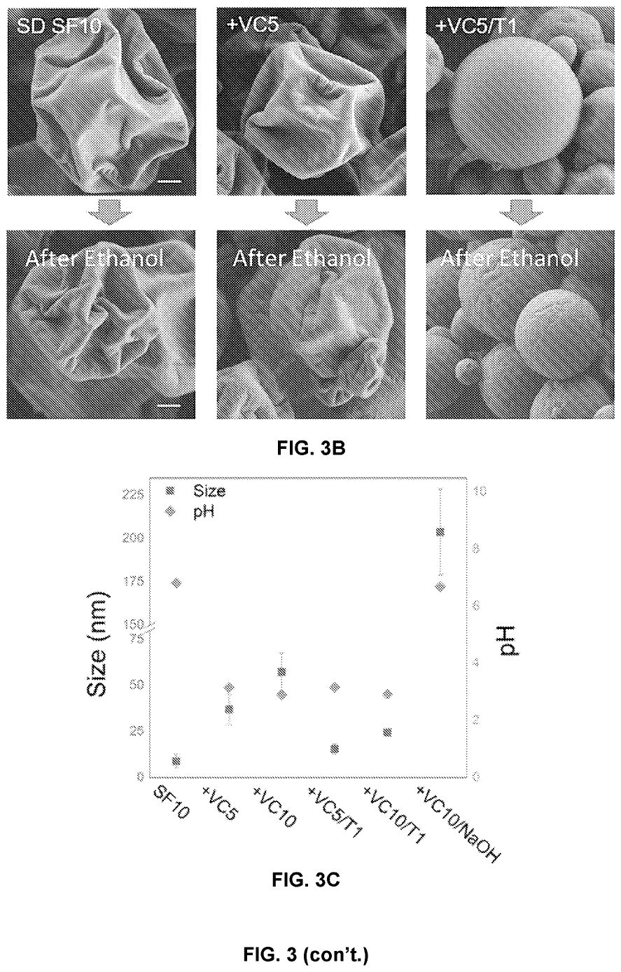

We prepared a series of silk-based microcapsules based on different compositions, generation methods and post-treatments and investigated their roles in morphology control (FIG. 3). For SFD microparticles, the addition of VC active increases the porosity on the surfaces (FIG. 3A). Pure SF microsphere presents a relatively smooth surface without visible pores, while adding 5% or 10% VC result in increased number of pores. It is worth noting that VC introduced acidity to the mixed suspension, which we tried neutralizing with sodium hydroxide. However, the SF/VC/NaOH microcapsules showed very porous and coarse surfaces with collapsed structures. Adding 1% of surfactant (Tween20) also resulted in partially porous coarse surfaces. Since the microdroplets of feed suspension were fast frozen during spray freeze drying, the distribution status of SF polymer chains in suspension was well kept for analysis. The spray drying process relies on the heated air-induced drying of microdroplets, which provides information on deformation resistance of polymer chains. As shown in FIG. 3B, the SD SF and SF/VC microparticles showed highly folded but dense surfaces. Since the feed suspensions contain 10% SF with or without 5% VC, where water molecules occupy most of the space in a microdroplet, the results indicate the SF polymer shrinks to compensate for the free space from water evaporation. The shrinkage also results in a denser surface due to drying induced assembly of SF polymer chains. Besides, adding surfactant leads to generation of dense, spherical microcapsules shown in FIG. 3B, it implies a more stable relaxation of SF in mixed suspension. Without wishing to be bound by any particular theory, it is believed that the acidity of VC tends to induce the agglomeration of the SF polymer chain. The excess protons may inhibit the ionization of hydrophilic domains in SF and lead to less uniform distribution in a microdroplet. To test that, we measured the size and pH of different suspensions using dynamic light scattering (FIG. 3C). The pH of neat 10% SF suspension is around 6.8 and drops to 3.1 and 2.9 when adding 5 and 10% of VC, respectively. Compensation using a base solution brings the pH back to 6.7. The size of neat 10% SF suspension (9 nm) increases to over 37 and 57 nm along with the dropping of pH, which indicates agglomeration of SF chains due to a weaker negative-negative repulsion. At low pH as 3.1 and 2.9, we introduced surfactant and successfully reduced the average size of SF to 16 and 25 nm. This implied that the addition of surfactant helps the relaxation of SF chains, while the addition of base solution compensates for the excess protons but leads to more agglomeration due to high ionic strength (average size around 204 nm).

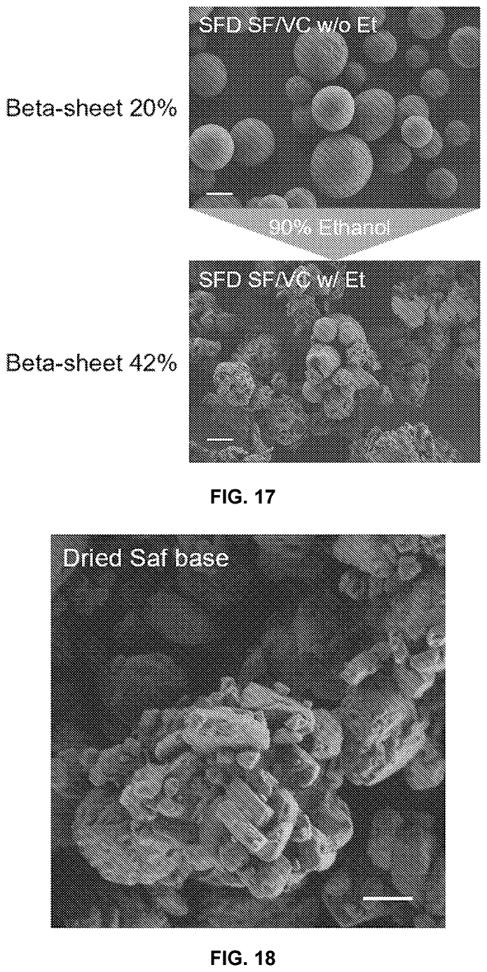

Moreover, the ethanol treated SD and SFD microparticles are shown in FIGS. 3B and 17. Due to the dense surfaces of drying induced assembly, SD microparticles showed a similar morphology which was retained after soaking in ethanol (FIG. 3B). The stability of SD microparticles in solvent treatment can contribute to fabrication of products with varied beta sheet, which affects dissolution and degradation rates for diverse applications. However, SFD microparticles were deformed and fused to each other after being treated with ethanol, which may be caused by the high contact area from their porous and sponge-like structures (FIG. 17).

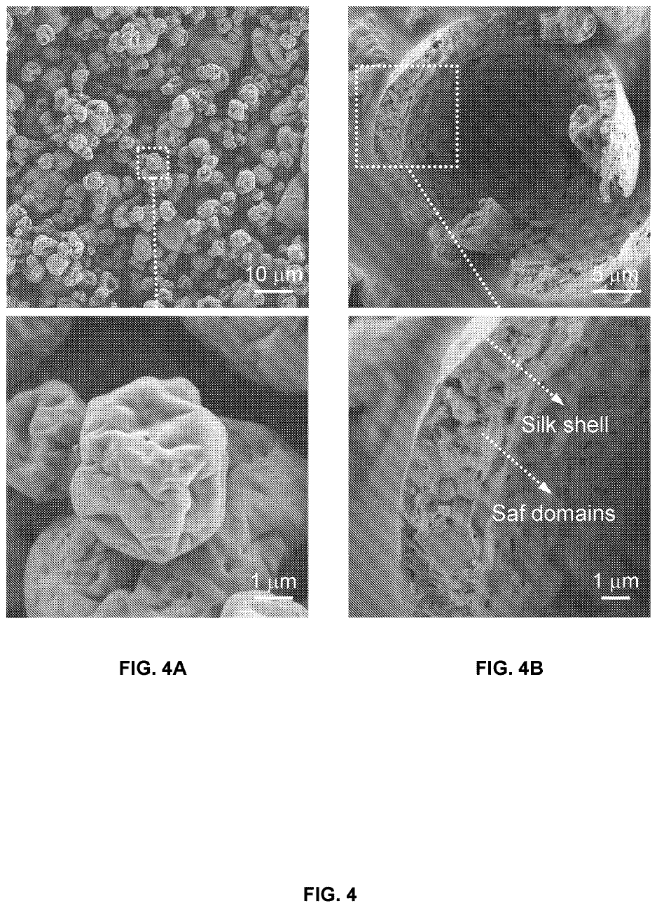

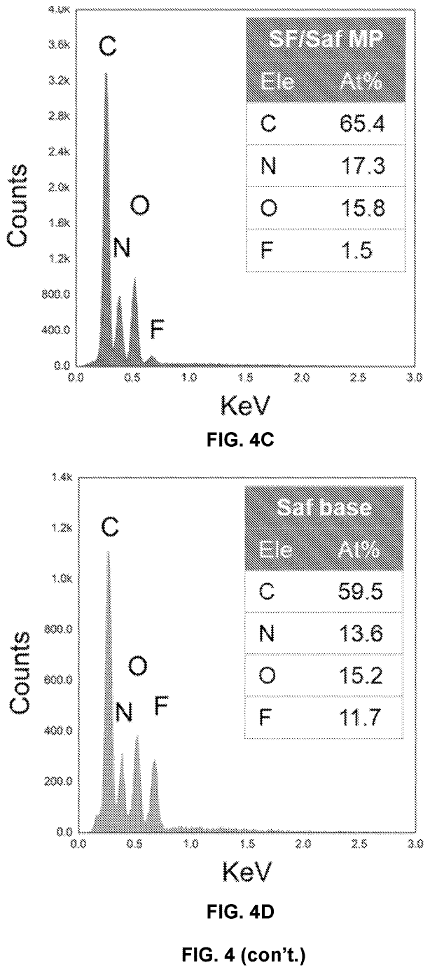

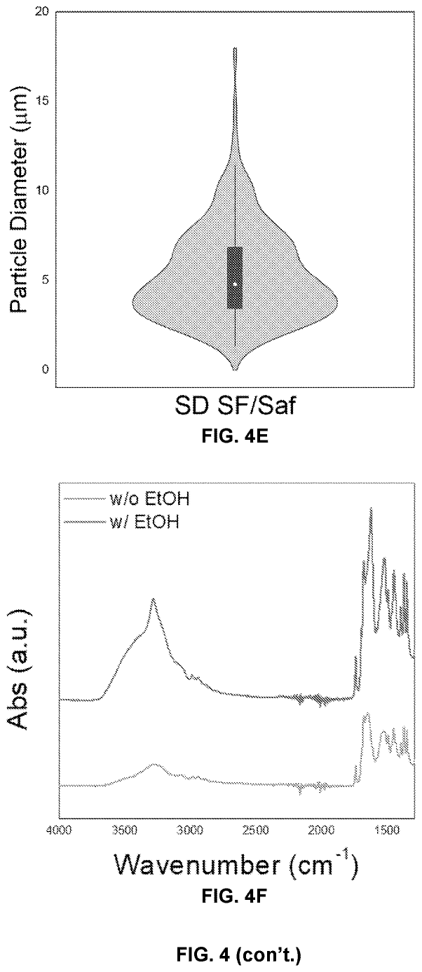

We further investigated silk-based encapsulation of an insoluble active—herbicide Saf. Saf is a stable (thermal decomposition T around 236° C.) solid powder with limited solubility in both water (2 g L⁻¹) and organic solvents. A Saf base was probe sonicated to around 1 μm in size with the presence of additives (FIG. 18). FIG. 4 presents a detailed morphological and structural characterization of ethanol treated SD SF/Saf microcapsules. The as-prepared microcapsules showed crumpled surfaces with a multi-domain hollow structure (FIGS. 4A and 4B). From the high-resolution SEM images, we could see a dense and continuous matrix with irregular micro-sized crystals exposed at the cross-section. In FIGS. 4C and 4D, the elemental analysis on the surface of microcapsules showed lower F atomic %(1.5%) compared with original atomic % (11.7%) in Saf base. The C and N atomic % of SF/Saf microcapsules was also close to that of neat SF (Table 1). These results support a multi-domain structure where Saf crystal pieces are embedded in a dense SF shell. The size of microcapsules is 5±3 μm (FIG. 4E). After ethanol treatment, the amide I region centered at 1647 shifted to 1622 cm⁻¹ as shown in FIG. 4F; all other peaks remained in the same positions, indicating a SF secondary structural transition from random coil to beta sheet.

TABLE 1
The elemental analysis of neat SF,
Saf base and SD SF/Saf microcaple.

| Elemental content (%) | C | N | O | F |
|---|---|---|---|---|
| Neat SF | 64.9 | 16.2 | 19.0 | 0 |
| Saf base | 59.5 | 13.6 | 15.2 | 11.7 |
| SD SF/Saf | 65.4 | 17.3 | 15.8 | 1.5 |

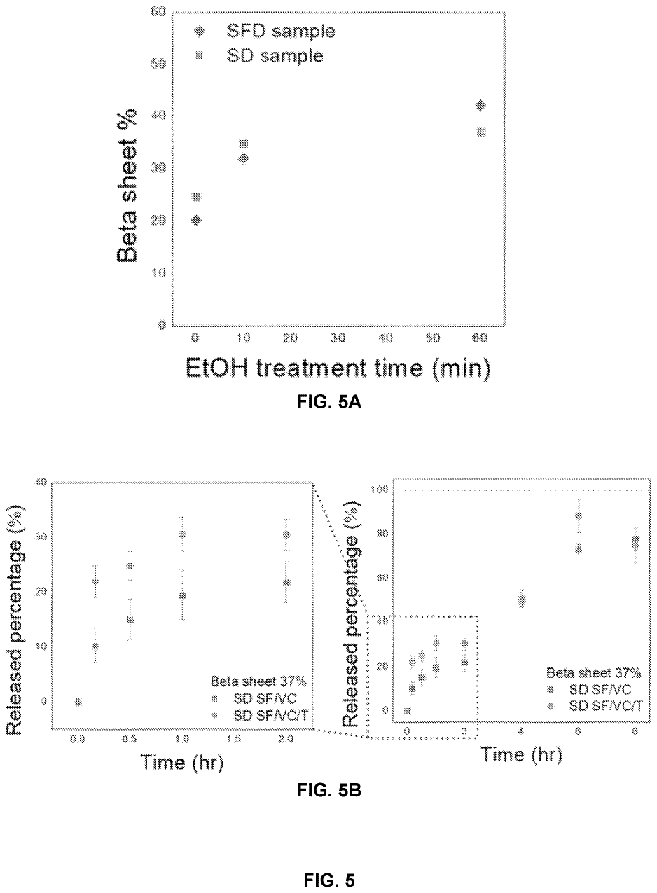

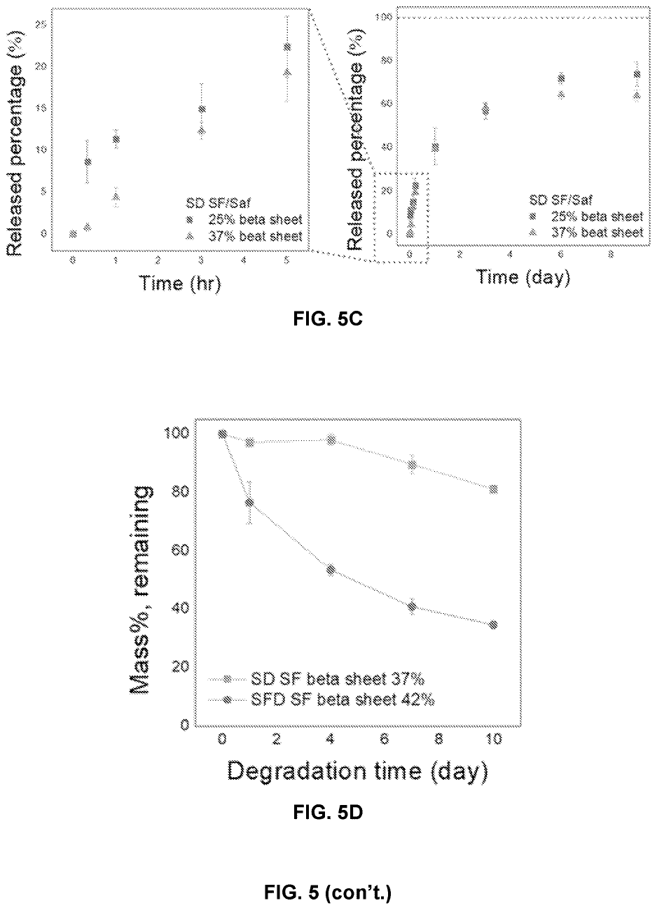

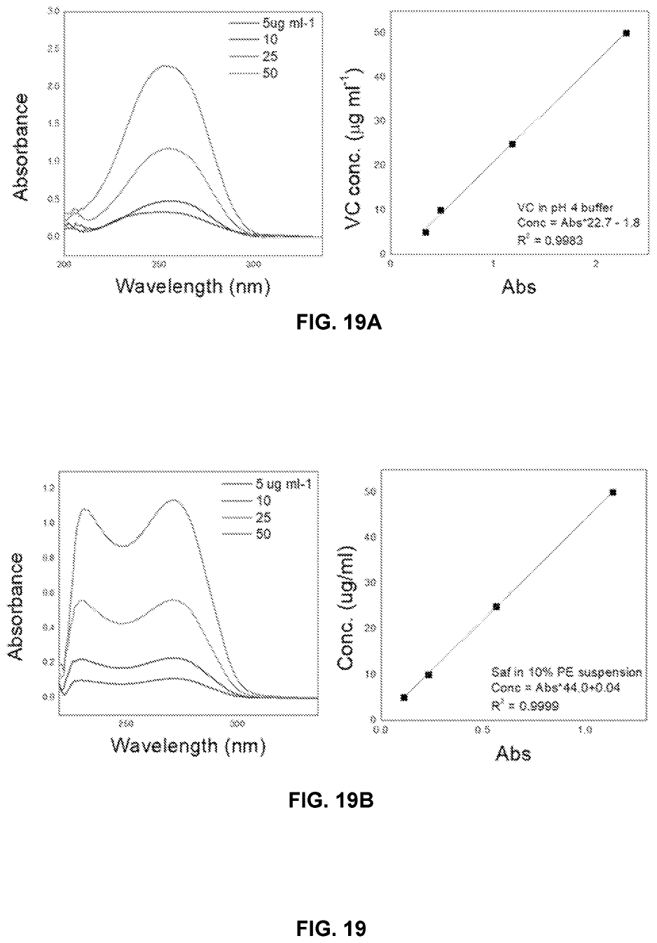

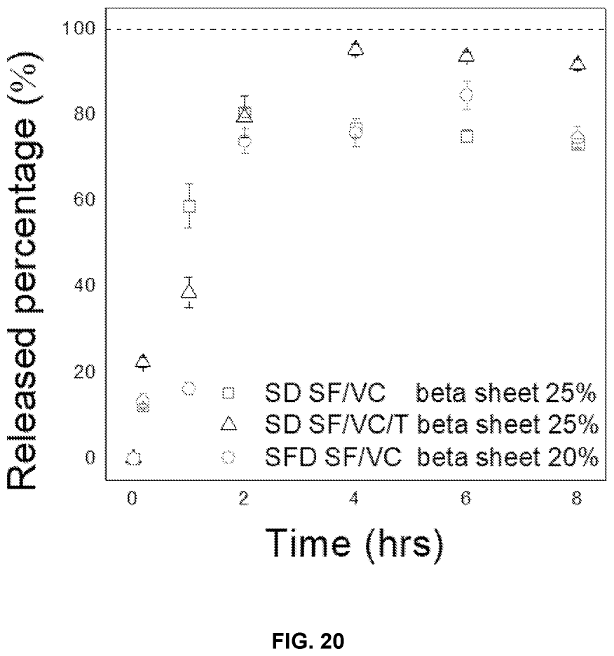

FIG. 5 presents the conformation of silk-based microcapsules after ethanol treatment and related release kinetics and degradation profiles. The SFD and SD SF microparticles were in contact with 90% ethanol for 0, 10, and 60 min followed with overnight drying. Their beta sheet percentages were calculated by Fourier self-deconvolution analysis of the amide I region in FTIR results (FIGS. 2F and 4F). In FIG. 5A, the beta sheet contents of SFD and SD samples increased gradually from 20 and 25% for at t=0 to 42 and 37% at t=60 mins. We then evaluated the release performance of microcapsules by monitoring the active release in solution media using UV-Vis spectroscopy. The concentration-absorption calibration curves of VC and Saf are shown in FIG. 19. The release kinetics of SD SF/VC microcapsules within 8 hrs are shown in FIG. 5B. The incorporation of SF retards the release of VC, while the burst release of SF/VC (10%) is lower than that of SF/VC/T (20%) (FIG. 5B inset): both reach equilibrium concentrations after 6 hrs. The release profiles show asymptote in concentrations, and the SF/VC sample released slower than SF/VC/T sample. This may be explained by the complex and crumpled morphology of SF/VC microcapsules that further retard the relaxation and dissociation of SF matrix. The SD and SFD SF/VC samples with lower beta sheet contents exhibit faster release and release equilibrium concentration in about 2 hrs (FIG. 20). Release kinetics of SD SF/Saf microcapsules with beta sheets 25 and 37% are shown in FIG. 5C. Without silk encapsulation, the burst release of free Saf crystals is near 25%. Microencapsulation by SF with 25% beta sheet drops the burst release to around 9%, and by SF with 37% beta sheet to 0.8%. The significant retardant of Saf release can be explained by the multi-domain structure and increased hydrophobicity of SF shell. The equilibrium released concentration of Saf is less than 80%, which implies that there are some remaining Saf crystals deeply embedded in SF shell. The biodegradability of ethanol treated SF microparticles was evaluated using a protease generated by protease IV from Streptomyces griseus (a soil-dwelling bacteria selected for open use microcapsules). Both ethanol treated SD and SFD SF sample degraded in a 10-day period (FIG. 5C). We anticipated that the SFD sample would degrade faster due to porous surfaces where protease can more easily diffuse inside.

### Silk Based Encapsulation of Lipophilic Active—Dibutyl Adipate

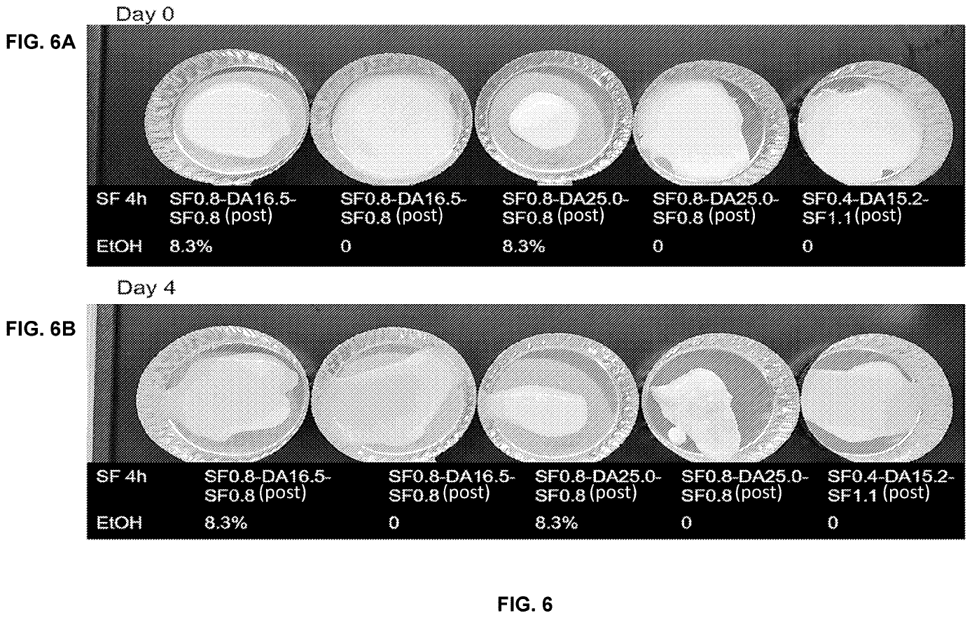

To explore the potential encapsulation of lipophilic actives by SF, we chose a solvent oil dibutyl adipate (DA) as the oily phase to prove the concept. The design was to emulsify aqueous SF and oily DA suspensions, where SF functions as an encapsulating shell around the oily active. Potential shell enhancement by increasing beta sheet content was also included, such as sonication-assisted aging and ethanol treatment. SF/DA emulsion study data includes shelf stability, microdroplet morphology and thermal stability evaluations. The SF/DA microemulsions were prepared by mixing SF aqueous suspension with DA under ultrasonication, followed with ethanol induced beta sheet transformation or aging for enhanced shell formation (for details see the methods section). Since the shearing stress during sonication tends to slowly gel SF, we pre-added 4 hrs boiled SF at low concentration as 0.8% before emulsification, followed with post addition of another 0.8% SF. The stability of SF/DA emulsions with different compositions is shown in FIG. 6. The aged emulsions with lower DA content remained relatively stable after 4 days, while emulsions with 25% DA became thicker. To overcome this gelation effect and to increase the stability of the dispersion without sacrificing the release, 2 hours boiled SF and different preparation methods could be used.

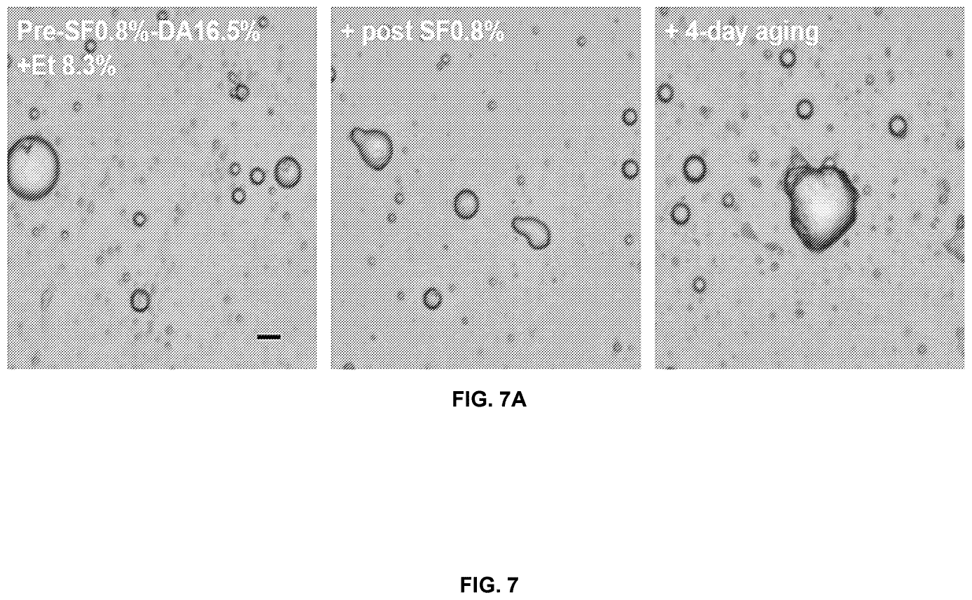

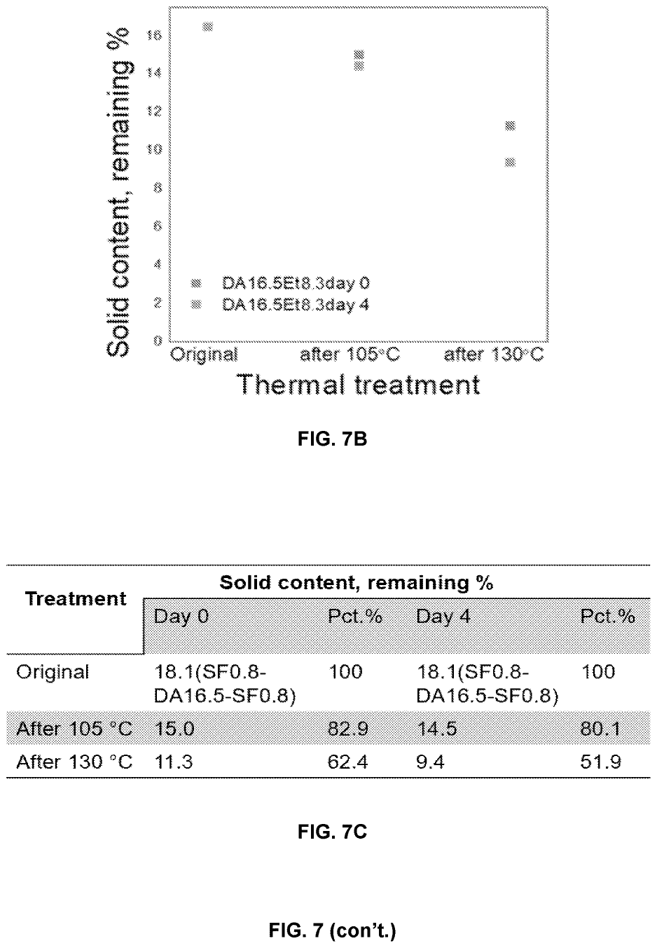

We performed a series of detailed characterization and thermal stability (encapsulation efficiency) evaluation of all five types of emulsions as shown in FIGS. 7-11, including morphologies after initial emulsification, after addition of complementary SF suspension, and after aging for a period of 4 days. The encapsulation capacity was evaluated by a two-stage annealing process, where the first stage at 105° C. for 2 hrs was used to test the total solid content and early thermal release. The effects of solid content percentage, pre- and post-addition of SF, addition of ethanol and ripening of emulsions are summarized below.

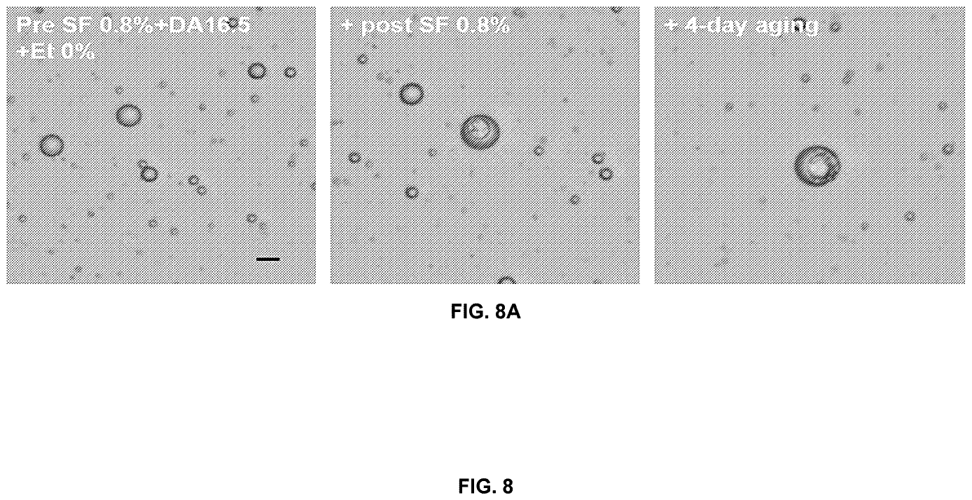

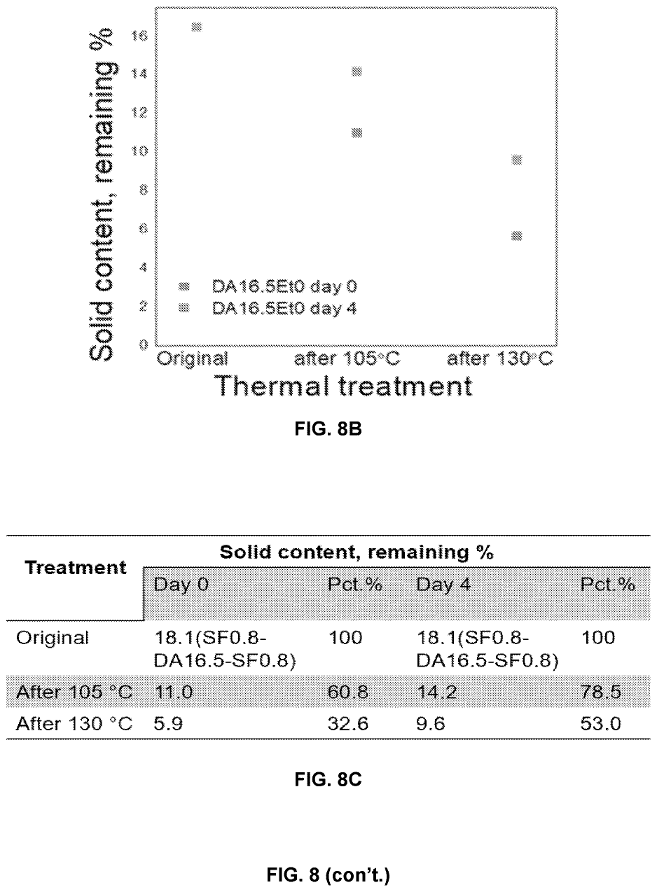

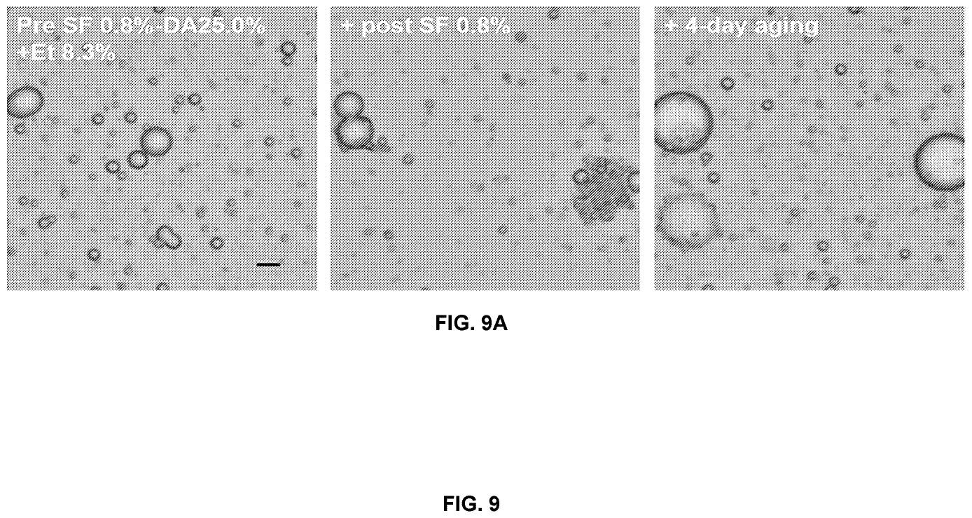

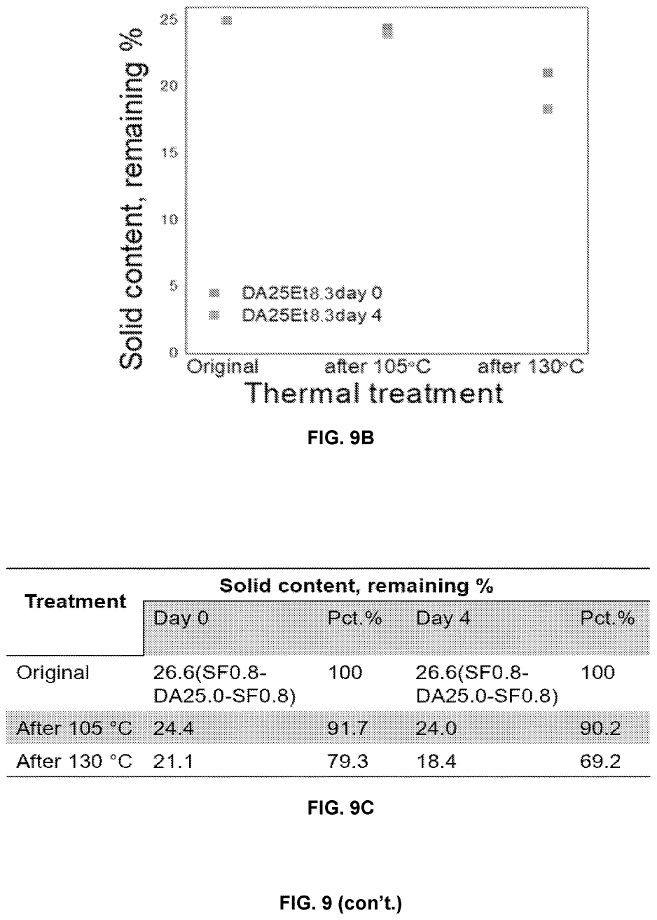

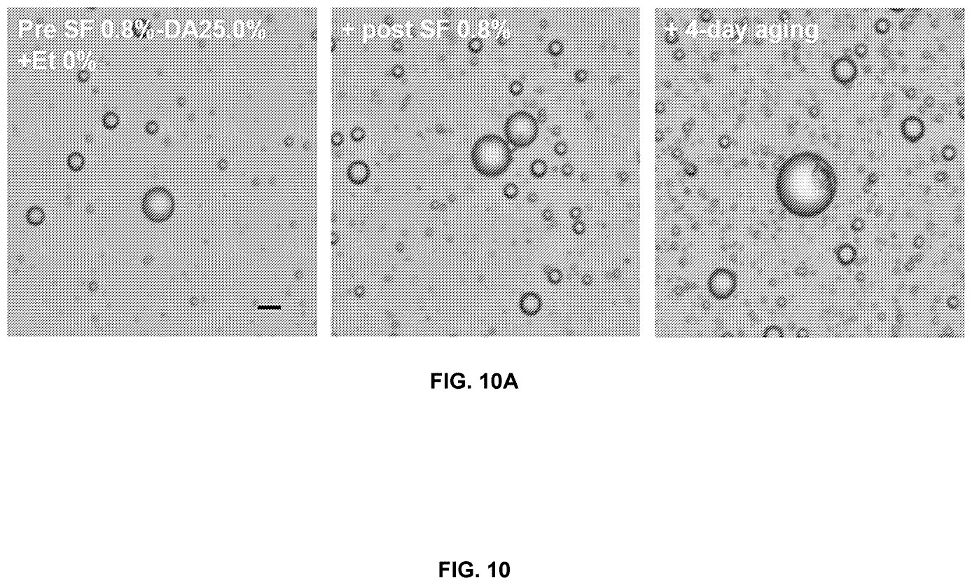

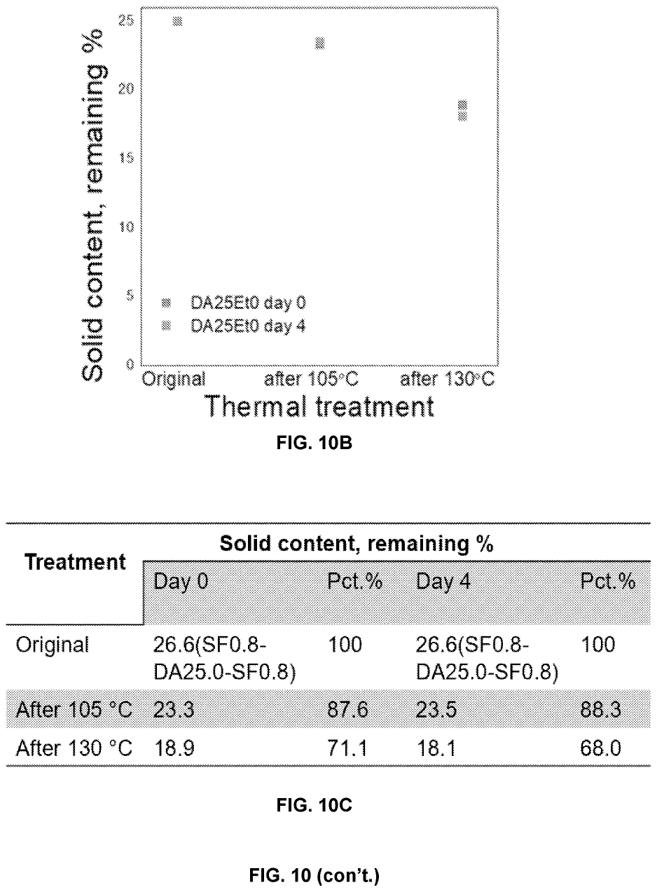

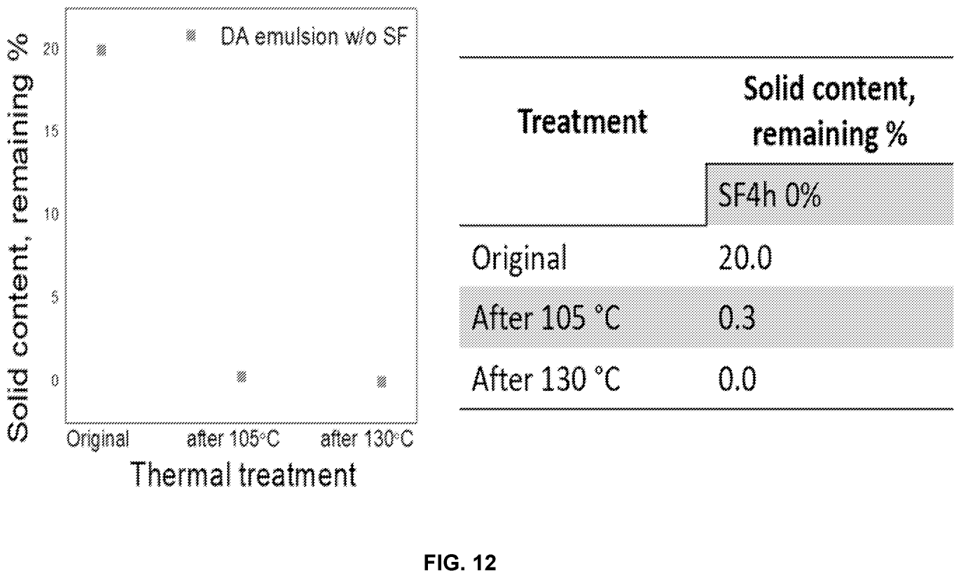

For SF/DA emulsions with different solid contents: Comparing results in FIG. 7 and FIG. 9, FIG. 8 and FIG. 10, the remaining solid contents increased along with the increasing of SF and DA wt %, indicating an enhanced encapsulation. The introduction of SF significantly improves the thermal stability of oily DA phase in all samples, the highest solid contents reach ca. 90% (percentage between the measured solid content over the theoretical solid content) after the first stage of thermal treatment, and ca. 80% after the second stage of thermal treatment (both from SF0.8+0.8%-DA25%-Et8.3% fresh emulsion). Without SF, almost all oily active evaporated after the first stage of thermal treatment (0.3% solid content remaining as shown in FIG. 12). This demonstrates the efficacy of silk to encapsulate active molecules, prevent thermal release, and reduce the volatility of the active. This phenomenon happens regardless of the addition of ethanol or ripening.

For SF/DA emulsions with different ethanol contents: Comparing results in FIG. 7 and FIG. 8, FIG. 9 and FIG. 10, the remaining solid contents slightly decrease with the addition of 8.3% of ethanol. It is possible that dropwise adding ethanol induced local gelling of free SF and lowered the encapsulation efficacy.

For SF/DA emulsions with different aging time: Comparing results in FIGS. 7-11, the emulsions with 10% ethanol showed a slight decrease in thermal stability, while those without ethanol showed a similar or slightly higher thermal stability. The aging after sonication lead to a slow assembly of beta sheets, which may have contributed to a better encapsulation of oily active. However, without wishing to be bound by any particular theory, the adding of ethanol may have triggered a random gelling of SF which adversely affected the distribution of the shell and core components, or the ethanol may have led to an early leaching of the oil out of the microcapsule during the warming of the emulsion.

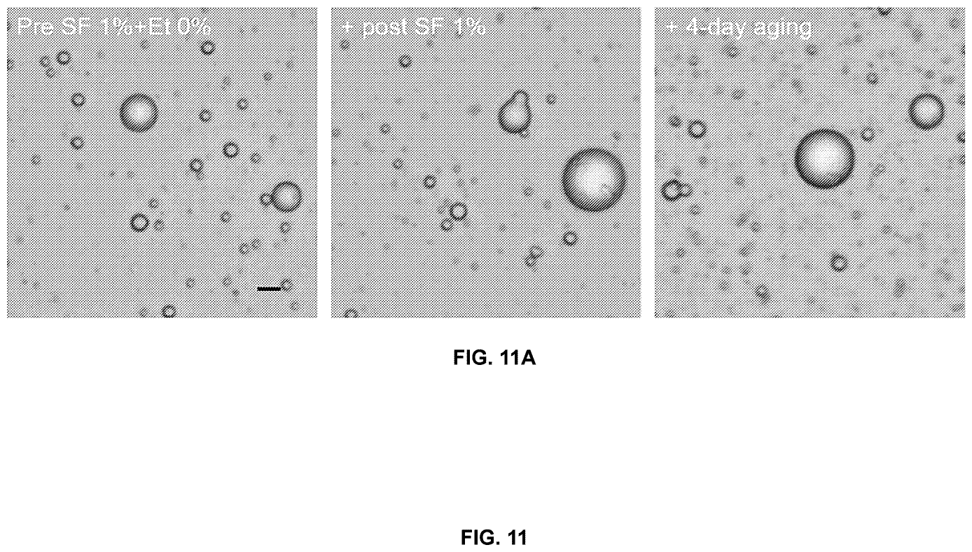

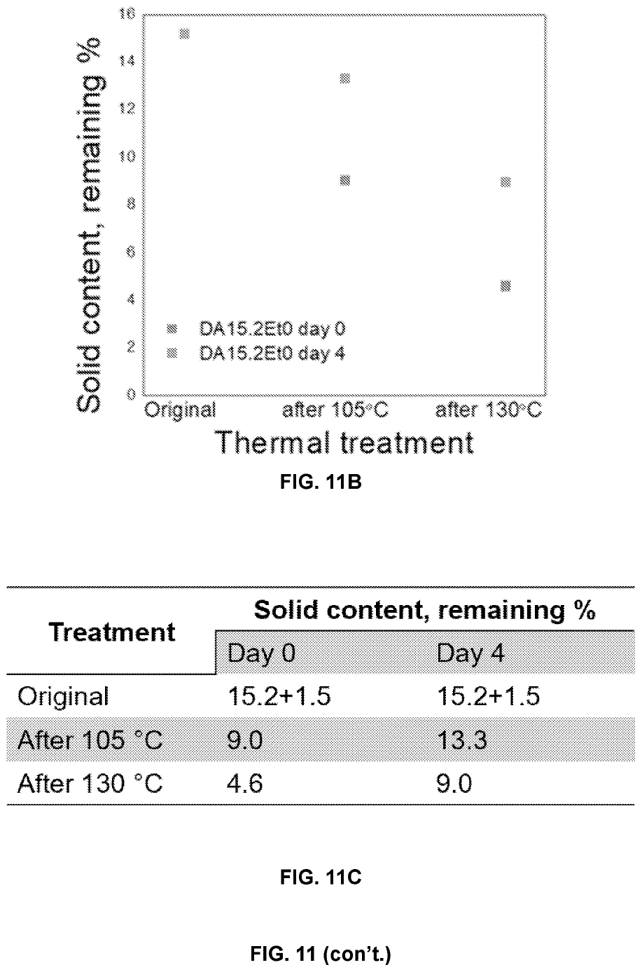

For SF/DA emulsions with different pre- and post-SF %: Comparing results in FIG. 8 and FIG. 11, the emulsions with 0.8+0.8% or 0.4+1.1% SF show a slight difference in thermal stability. For a similar final load, less SF before and more after the sonication induced emulsification seemed not to lead to more uniform encapsulation.

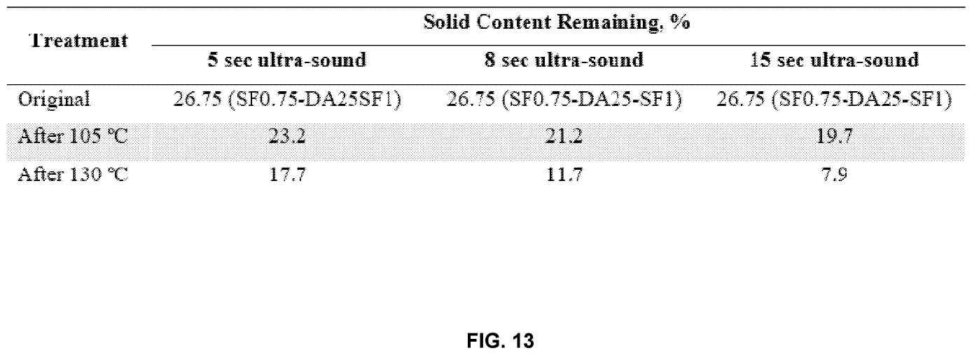

For SF/DA emulsions with different sonication time: Comparing results in FIG. 13, for the same formulation composition, the emulsions with shorter sonication time showed higher solid content and remaining content after heat treatment. This effect could have originated with the difference in terms of particles size of the microcapsules, which are bigger at shorter emulsification time. The aqueous dispersions obtained were all stable for several days without gelation.

### Silk Based Encapsulation of Lipophilic Active—Tinosorb® S

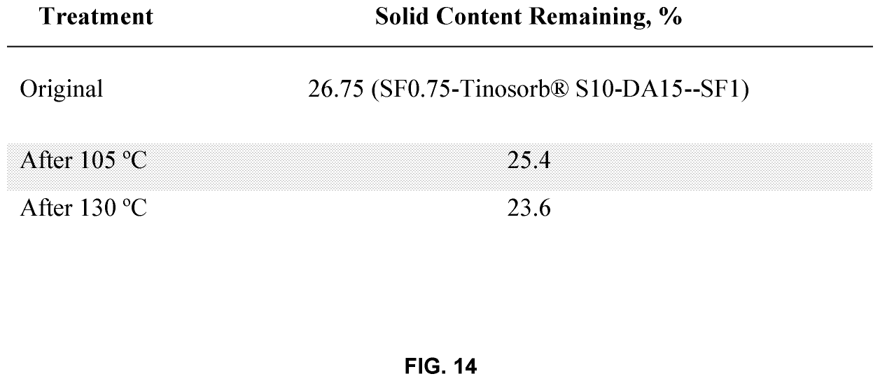

Tinosorb® S (bis-ethylhexyloxyphenol methoxyphenyl triazine) is a non-water-soluble UV-filter which is solid at room temperature. To encapsulate it in silk, the active was first dissolved in DA (weight ratio Tinosorb® S/DA: 60:40) and then encapsulated in silk via an emulsification process. FIG. 14 shows that the remaining content after different thermal treatment is really high.

Some embodiments of this invention are described herein, including the best mode known to the inventors for carrying out the invention. Of course, variations on these described embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

We claim:

1. A microcapsule comprising a silk fibroin polypeptide and one or more active payload materials, wherein the one or more active payload materials is present in an amount in the range of about 10% to about 98% by weight, based on solid weight of the microcapsule.

2. The microcapsule according to claim 1, wherein said microcapsule is a core-shell microcapsule having a shell of silk fibroin polypeptide and a core composed of the one or more payload materials.

3. The microcapsule according to claim 2, wherein the one or more active payload materials is dissolved in a non-aqueous solvent that is immiscible with water.

4. The microcapsule according to any one of claims 1 to 3, wherein said microcapsule is a microsphere with at least one active substance is dispersed within the silk fibroin polypeptide payload.

5. The microcapsule according to any one of claims 1 to 4, wherein the microcapsule is a hollow sphere.

6. The microcapsule of according to any of claims 1 to 5, wherein the microcapsule is a porous sphere, optionally having a porosity in the range of about 1% and 95%.

7. The microcapsule of any one of claims 1 to 5, having an average diameter in the range of about 1 μm to 50 μm, such as in the range of about 1 μm to 40 μm, or 2 μm to 30 μm, or 3 μm to 20 μm, preferably 3 μm to 15 μm.

8. The microcapsule of any one of claims 1 to 5, having an average diameter in the range of about 1 μm to 50 μm, such in the range of about 5 μm to 50 μm, or 10 μm to 40 μm, preferably 15 μm to 35 μm.

9. The microcapsule of any one of claims 1 to 8, wherein the ratio of the silk fibroin polypeptide to the active payload material is from about 10:1 to about 1:50, such as about 3:1 to about 1:50, about 3:1 to about 1:25, about 3:1 to about 1:10, about 3:1 to about 1:5, or about 3:1 to about 1:3.

10. The microcapsule of any one of claims 1 to 9, wherein the one or more active payload materials is present in the range of about 10% to 98%, such as about 20% to 95%, preferably above 50% and more preferably above 70% by weight based on solid weight of the microcapsule.

11. The microcapsule of any one of claims 1 to 9, wherein the one or more active payload materials is present in the range of about 20% to 50%, such as about 25% to 50%, or about 25% to 40%, or about 25% to 35%, by weight based on solid weight of the microcapsule.

12. The microcapsule of any one of claims 1 to 11, wherein the active payload material is hydrophilic.

13. The microcapsule of any one of claims 1 to 11, wherein the active payload material is lipophilic.

14. The microcapsule of any one of claims 1 to 11, wherein the active payload material is solid at room temperature and water insoluble.

15. The microcapsule of any one of claims 1 to 11, wherein the active payload material is solid at room temperature and water insoluble and dissolved in a non-aqueous solvent that is immiscible with water.

16. The microcapsule of any one of claims 1 to 15, wherein the one or more active payload materials is selected from pesticides, synergists, plant health agents, repellants, biocides, phase-change materials, pharmaceuticals, cosmetic ingredients (like fragrances, perfumes, vitamins, essential oils, plant extracts), nutrients, food additives (like vegetable oils, marine oils, vitamins, aromas, antioxidants, essential oils, plant extracts), pheromones, catalysts, and combinations thereof.

17. The microcapsule of any one of claims 1 to 16, wherein the silk fibroin polypeptide has a beta sheet content in the range of 10% to 50%.

18. The microcapsule of any one of claims 1 to 17, wherein the microcapsules has been treated with methanol, ethanol, propanol, glycol, acetone, salt solution or any other treatment (such as a physical process using ultra-sound) to increase the percentage of beta sheet content.

19. The microcapsule of any one of claims 1 to 18, wherein the silk fibroin polypeptide has an average molecular weight of between about 15 kDa and about 400 kDa or about 50 kDa to 300 kDa.

20. The microcapsule of any one of claims 1 to 19, wherein the silk fibroin polypeptide is not crosslinked.

21. The microcapsule of any one of claims 1 to 20, wherein the silk fibroin polypeptide is extracted from Bombyx mori cocoons.

22. The microcapsule of any one of claims 1 to 20, wherein the silk fibroin polypeptide is obtained by fermentation.

23. A method of preparing a microcapsule of any one of claims 1 to 22, the method comprising:
(a) mixing a silk fibroin polypeptide and one or more of active payload materials in an aqueous solution to obtain a mixture:
(b) feeding and atomizing said mixture into a drying chamber to obtain droplets; and
(c) drying said droplets to microcapsules.

24. A method of preparing a microcapsule of any one of claims 1 to 22, the method comprising:
(a) mixing a silk fibroin polypeptide and one or more of active payload materials in an aqueous solution to obtain a mixture:
(b) feeding and atomizing said mixture into a cold fluid to obtain frozen droplets; and
(c) drying said droplets to microcapsules.

25. The method according to claim 23 or claim 24, further comprising (d) treating the microcapsules to increase the beta sheet content.

26. The method of claim 25, wherein the treating is contacting the microcapsules with methanol, ethanol, propanol, glycol, acetone, salt solution, and/or treating is subjecting the microcapsules to a physical process (such as ultra-sound).

27. A method of preparing a microcapsule of any one of claims 1 to 22, the method comprising:
(a) creating an organic phase composed of the one or more of active payload materials, optionally dissolved in a non-aqueous solvent that is immiscible with water; and
(b) emulsifying the organic phase in water containing silk fibroin polypeptide to obtain microcapsules.

28. The method according to claim 27, further comprising (c) treating the microcapsules to increase the beta sheet content.

29. The method of claim 28, wherein the treating is contacting the microcapsules with methanol, ethanol, propanol, glycol, acetone, and/or salt solution, and/or treating is subjecting the microcapsules to a physical process.

30. The method according to claim 29, wherein the physical process is ultra-sound.

31. A formulation comprising microcapsules according to any of claims 1 to 22 or prepared according to claims 23 to 30, wherein said microcapsules are present as dispersed particles in an aqueous medium.

32. The formulation according to claim 31, wherein said formulation comprises 1 to 50 wt %, preferably 5 to 45 wt %, more preferably 10 to 40 wt % of said one or more active substances.

33. Use of microcapsules according to any of claims 1 to 22 or prepared according to claims 23 to 30 or the formulation according to claims 31 or 32 in agrochemical applications (e.g. crop protection, agricultural non-crop applications, seed treatment), pharmaceutical applications, public health, personal care applications, construction applications, textile applications, human or animal nutrition applications, chemical process applications, adhesives and sealants, paints and coatings, building and construction materials, self-healing materials, tobacco industry, or household applications.

34. The use according to claim 33, wherein the agrochemical applications are crop protection, agricultural non-crop applications, and/or seed treatment.

35. The use according to claim 33, wherein the personal care applications are cosmetic applications.

36. A method for controlling phytopathogenic fungi and/or undesired plant growth and/or undesired attack by insects or mites and/or for regulating the growth of plants, where microcapsules according to any of claims 1 to 22 or prepared according to claims 23 to 30 or the formulation according to claims 31 or 32 are applied to the plants and/or the soil.

37. The method according to claim 36, wherein the application to the plants and/or the soil comprises allowing the microcapsules or formulation to act on the pests, their habitat, the plants to be protected from the pests, the soil, and/or on undesired plants and/or the useful plants and/or their habitat.

38. A method of improving plant growth, the method comprising applying an effective amount microcapsules according to any of claims 1 to 22 or prepared according to claims 23 to 30, or the formulation according to claims 31 or 32 to seed bed, soil, and/or plant, such as plant foliage.

39. Seed coatings comprising microcapsules according to any of claims 1 to 22 or prepared according to claims 23 to 30.


SEQUENCE LISTING (1 sequence)

SEQ ID NO: 1 - protein, 6 aa, synthetic construct
GAGAGS